(19) United States
(12) Patent Application Publication
(10) Pub. No.: US 2009/0203980 A1
(43) Pub. Date: Aug. 13, 2009

(54) SENSORS EMPLOYING COMBINATORIAL ARTIFICIAL RECEPTORS

(76) Inventors: Robert E. Carlson, Minnetonka, MN (US); Rachel L. Weller Roska, Minneapolis, MN (US); Stephen A. Brose, St. Paul, MN (US)

Correspondence Address: MERCHANT & GOULD PC, P.O. BOX 2903, MINNEAPOLIS, MN, 55402-0903, US

(21) Appl. No.: 12/325,773

(22) Filed: Dec. 1, 2008

Related U.S. Application Data
(60) Provisional application No. 60/991,028, filed on Nov. 29, 2007.
(60) Provisional application No. 61/125,796, filed on Apr. 28, 2008.
(60) Provisional application No. 61/128,372, filed on May 20, 2008.
(60) Provisional application No. 61/112,507, filed on Nov. 7, 2008.

(51) Int. Cl. A61B 5/145

(52) U.S. Cl. 600/365

(57) ABSTRACT

The present invention relates to sensors including artificial receptors and methods of using them. In an embodiment, the present invention includes an artificial receptor as a component of a receptor system including a ligand permeable interface that isolates the artificial receptor from certain components of the surrounding environment. In an embodiment, the present invention includes an artificial receptor and a competitor against a ligand of interest.

In an embodiment, the present invention includes a competitive artificial receptor as a component of a detector system including a semipermeable membrane that isolates the competitive artificial receptor from certain components of the surrounding environment. This embodiment also includes the competitor and a detector operatively coupled to the competitive artificial receptor. The detector produces a signal indicating binding of the competitor and/or the ligand of interest to the artificial receptor. The detector system is configured so that the competitor is retained in the environs of the artificial receptor at least in part by the ligand permeable interface.

### CROSS REFERENCE TO RELATED APPLICATIONS

The present application claims priority to U.S. Provisional Patent Application Nos. 60/991,028, filed Nov. 29, 2007, 61/125,796, filed Apr. 29, 2008, 61/128,372, filed May 20, 2008, and 61/112,507, filed Nov. 7, 2008. Each of these patent applications is incorporated herein by reference in its entirety.

### FIELD OF THE INVENTION

The present invention relates to sensors including artificial receptors and methods of using them. In an embodiment, the present invention includes an artificial receptor as a component of a receptor system including a ligand permeable interface that isolates the artificial receptor from certain components of the surrounding environment. In an embodiment, the present invention includes an artificial receptor and a competitor against a ligand of interest.

In an embodiment, the present invention includes a competitive artificial receptor as a component of a detector system including a semipermeable membrane that isolates the competitive artificial receptor from certain components of the surrounding environment. This embodiment also includes the competitor and a detector operatively coupled to the competitive artificial receptor. The detector produces a signal indicating binding of the competitor and/or the ligand of interest to the artificial receptor. The detector system is configured so that the competitor is retained in the environs of the artificial receptor at least in part by the ligand permeable interface.

### BACKGROUND

Diabetes is one of the leading causes of morbidity and mortality. In addition to quality of life issues, diabetes takes an enormous economic toll both on the individual and on society. Although diabetes is presently not curable, accurate monitoring of blood glucose levels when combined with insulin therapy dramatically improves on both lifestyle and lifespan. For diabetic patients, glucose levels must be checked or monitored several times throughout the day so that insulin may be periodically administered in order to maintain the glucose concentration at a normal level.

In one popular method, the glucose level is monitored by first obtaining a sample of blood from finger-pricking. Over the long-term, the requirement that the diabetic must prick their finger multiple times a day for a blood sample leads to less than ideal measurement frequency and, as a result, out of range blood glucose levels. Furthermore, glucose levels often fluctuate throughout the day. Therefore, even diabetic patients who are otherwise consistent in checking their glucose levels at regular intervals throughout the day may be unaware of periods wherein their glucose levels are unacceptably low or high.

Over the past several decades, there have been efforts to build a useful in vivo glucose sensor. In such implantable devices, an electrochemical sensor is embedded beneath the skin of the patient. None of these efforts have been successful. Therefore, there remains a need for an implantable glucose measurement system.

### SUMMARY

The present invention relates to sensors including artificial receptors and methods of using them. In an embodiment, the present invention includes an artificial receptor as a component of a receptor system including a ligand permeable interface that isolates the artificial receptor from certain components of the surrounding environment. In an embodiment, the present invention includes an artificial receptor and a competitor against a ligand of interest.

In an embodiment, the present invention includes a competitive artificial receptor as a component of a detector system including a semipermeable membrane that isolates the competitive artificial receptor from certain components of the surrounding environment. This embodiment also includes the competitor and a detector operatively coupled to the competitive artificial receptor. The detector produces a signal indicating binding of the competitor and/or the ligand of interest to the artificial receptor. The detector system is configured so that the competitor is retained in the environs of the artificial receptor at least in part by the ligand permeable interface.

In an embodiment, the present invention relates to an artificial receptor system. This embodiment can include a molecular competitor, an artificial receptor, a ligand permeable interface, and a detector. The molecular competitor competes against binding of a ligand of interest. The artificial receptor is configured to bind a ligand of interest and the competitor. Binding of the ligand of interest competes with binding of the competitor. The artificial receptor includes a plurality of different building block molecules independently covalently coupled to a solid support. The ligand permeable interface is permeable to the ligand of interest but retains an effective amount of the competitor. The ligand permeable interface and the artificial receptor at least partially define a chamber. The competitor and the artificial receptor are in the chamber. The detector is operatively coupled to the artificial receptor. The detector is configured to produce indicia of presence or concentration of the ligand of interest.

In an embodiment, the present invention relates to a detector system. This detector system includes a competitor, an artificial receptor, a semipermeable membrane, and a detector. The competitor includes an analog of a ligand of interest covalently coupled to a macromolecule. The artificial receptor is configured to bind a ligand of interest and the competitor. Binding of the ligand of interest competes with binding of the competitor. The artificial receptor includes a plurality of different building block molecules independently covalently coupled to a solid support in a region. The region is a contiguous portion of the surface of the solid support. The different building block molecules are distributed randomly throughout the contiguous region. The semipermeable membrane is permeable to the ligand of interest but retains an effective amount of the competitor. The semipermeable membrane and the artificial receptor at least partially define a chamber. The competitor and the artificial receptor are in the chamber. The detector is operatively coupled to the artificial receptor and configured to, in response to binding of the competitor or the ligand of interest to the artificial receptor, produce indicia of presence or concentration of the ligand of interest.

The present invention also includes a method of determining a level of a ligand of interest. In an embodiment, the method includes implanting in a subject the present detector system. In an embodiment, the method includes implanting in a subject the present artificial receptor system. The method also includes retrieving from the system indicia of presence or concentration of the ligand of interest.

### BRIEF DESCRIPTION OF THE DRAWINGS

FIG. 1 schematically illustrates an embodiment of the present system including an artificial receptor and a detector.

FIG. 2 schematically illustrates an embodiment of the present system configured for binding and detecting glucose and including an artificial receptor and a detector.

FIG. 3 schematically illustrates an embodiment of the present glucose sensor including antenna and circuit for receiving and transmitting radio signal.

FIG. 4 schematically illustrates a glucose sensor configured to communicate low, safe, and high glucose read-outs.

FIG. 5 is a bar graph depicting the difference in binding on an N₂₉n₁₋₂ microarray between a competitor agent in competition with 100,000× excess glucose and the competitor agent alone. A positive difference value indicates competition (y-axis, competition binding in the presence of glucose minus competitor binding alone).

FIG. 6 illustrates the results obtained for binding of the glucose-dendrimer conjugate to certain candidate artificial receptors, with the highs and lows for receptors including three to six building blocks indicating diverse binding useful for obtaining the desired receptor.

FIG. 7 shows fluorescence images of artificial receptors that have been incubated with four different competitors. The relative intensity of the spots reflects the relative binding between the receptor and the competitor agent.

FIGS. 8 and 9 illustrate the results of competition for candidate receptors. In the study reported in FIG. 8, the labeled conjugate of glucose and dendrimer competed against the unlabeled conjugate for each of the candidate artificial receptors. FIG. 9 illustrates the results of an experiment in which the labeled conjugate of glucose and dendrimer competed with glucose.

FIG. 10 is a line graph showing glucose titration competition curves for selected binding environments from N₉n₁₋₉ microarrays.

### DETAILED DESCRIPTION OF THE PREFERRED EMBODIMENT

### Definitions

A combination of building blocks immobilized on, for example, a support can be a candidate artificial receptor, a lead artificial receptor, or a working artificial receptor. That is, a heterogeneous building block spot on a slide or a plurality of building blocks coated on a tube, well, bead, or self assembled monolayer can be a candidate artificial receptor, a lead artificial receptor, or a working artificial receptor. A candidate artificial receptor can become a lead artificial receptor, which can become a working artificial receptor.

As used herein the phrase “candidate artificial receptor” refers to an immobilized combination of building blocks that can be tested to determine whether or not a particular ligand of interest binds to that combination. In an embodiment, the candidate artificial receptor can be a heterogeneous building block spot on a slide or a plurality of building blocks coated on a tube or well.

As used herein the phrase “lead artificial receptor” refers to an immobilized combination of building blocks that binds glucose at a predetermined concentration of glucose, for example at 10, 1, 0.1, or 0.01 μg/ml, or at 1, 0.1, or 0.01 ng/ml. In an embodiment, the lead artificial receptor can be a heterogeneous building block spot on a slide or a plurality of building blocks coated on a tube or well.

As used herein the phrase “working artificial receptor” refers to a combination of building blocks that binds glucose with a selectivity and/or sensitivity effective for categorizing or identifying glucose. That is, binding to that combination of building blocks describes glucose as belonging to a category of test ligands or as being a particular test ligand. A working artificial receptor can, for example, bind glucose at a concentration of, for example, 100, 10, 1, 0.1, 0.01, or 0.001 ng/ml. In an embodiment, the working artificial receptor can be a heterogeneous building block spot on a slide or a plurality of building blocks coated on a tube, well, slide, or other support or on a scaffold.

As used herein the phrase “working artificial receptor complex” refers to a plurality of artificial receptors, each a combination of building blocks, that binds glucose with a pattern of selectivity and/or sensitivity effective for categorizing or identifying glucose. That is, binding to the several receptors of the complex describes glucose as belonging to a category of sugars, for example or as being glucose and not another sugar like fucose, or any other glucose analogue. The individual receptors in the complex can each bind glucose at different concentrations or with different affinities. For example, the individual receptors in the complex each bind glucose at concentrations of 100, 10, 1, 0.1, 0.01 or 0.001 ng/ml. In an embodiment, the working artificial receptor complex can be a plurality of heterogeneous building block spots or regions on a slide; a plurality of wells, each coated with a different combination of building blocks; or a plurality of tubes, each coated with a different combination of building blocks.

As used herein, the phrase “significant number of candidate artificial receptors” refers to sufficient candidate artificial receptors to provide an opportunity to find a working artificial receptor, working artificial receptor complex, or lead artificial receptor. As few as less than about 1000 candidate artificial receptors can be a significant number for finding working artificial receptor complexes suitable for distinguishing two sugars (e.g., glucose and galactose). In other embodiments, a significant number of candidate artificial receptors can include about 1,000 candidate artificial receptors, about 10,000 candidate artificial receptors, about 100,000 candidate artificial receptors, or more.

As used herein, the term “building block” refers to a molecular component of an artificial receptor including portions that can be envisioned as or that include one or more linkers, one or more core carriers, and one or more recognition elements (glucose analogues). In an embodiment, the building block includes a linker, a framework, and one or more recognition elements.

As used herein, the term “linker” refers to a portion of or functional group on a building block that can be employed to or that does couple the building block to a support, for example, through covalent link, ionic interaction, electrostatic interaction, or hydrophobic interaction.

As used herein, the term “framework” refers to a portion of a building block including the linker or to which the linker is coupled and to which one or more recognition elements are coupled.

As used herein, the term “recognition element” refers to a portion of a building block coupled to the framework but not covalently coupled to the support. Although not limiting to the present invention, the recognition element can provide or form one or more groups, surfaces, or spaces for interacting with the ligand.

As used herein, the phrase “plurality of building blocks” refers to two or more building blocks of different structure in a mixture, in a kit, or on a support or scaffold. Each building block has a particular structure, and use of building blocks in the plural, or of a plurality of building blocks, refers to more than one of these particular structures. Building blocks or plurality of building blocks does not refer to a plurality of molecules each having the same structure.

As used herein, the phrase “combination of building blocks” refers to a plurality of building blocks that together are in a spot, region, or a candidate, lead, or working artificial receptor. A combination of building blocks can be a subset of a set of building blocks. For example, a combination of building blocks can be one of the possible combinations of 2, 3, 4, 5, or 6 building blocks from a set of N (e.g., N=10-200) building blocks.

As used herein, the phrases “homogenous immobilized building block” and “homogenous immobilized building blocks” refer to a support or spot having immobilized on or within it only a single building block.

As used herein, the phrase “activated building block” refers to a building block activated to make it ready to form a covalent bond to a functional group, for example, on a support. A building block including a carboxyl group can be converted to a building block including an activated ester group, which is an activated building block. An activated building block including an activated ester group can react, for example, with an amine to form a covalent bond.

As used herein, the term “naïve” used with respect to one or more building blocks refers to a building block that has not previously been determined or known to bind to a ligand of interest. For example, the recognition element(s) on a naïve building block has not previously been determined or known to bind to a ligand of interest. A building block that is or includes a known ligand (e.g., GMI) for a particular protein (test ligand) of interest (e.g., cholera toxin) is not naïve with respect to that protein (test ligand).

As used herein, the term “immobilized” used with respect to building blocks coupled to a support refers to building blocks being stably oriented on the support so that they do not migrate on the support or release from the support. Building blocks can be immobilized by covalent coupling, by ionic interactions, by electrostatic interactions, such as ion pairing, or by hydrophobic interactions, such as van der Waals interactions.

As used herein a “region” of a support, tube, well, or surface refers to a contiguous portion of the support, tube, well, or surface. Building blocks coupled to a region can refer to building blocks in proximity to one another in that region.

As used herein, the term “support” refers to a solid support that is, typically, macroscopic.

As used herein, a “bulky” group on a molecule is larger than a moiety including 7 or 8 carbon atoms.

As used herein, a “small” group on a molecule is hydrogen, methyl, or another group smaller than a moiety including 4 carbon atoms.

As used herein, the term “lawn” refers to a layer, spot, or region of functional groups on a support, for example, at a density sufficient to place coupled building blocks in proximity to one another. The functional groups can include groups capable of forming covalent, ionic, electrostatic, or hydrophobic interactions with building blocks.

As used herein, the term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₁₂ for straight chain, C₁-C₆ for branched chain). Likewise, cycloalkyls can have from 3-10 carbon atoms in their ring structure, for example, 5, 6 or 7 carbons in the ring structure.

The term “alkyl” as used herein refers to both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl alkyl, or an aromatic or heteroaromatic moiety. The moieties substituted on the hydrocarbon chain can themselves be substituted, if appropriate. For example, the substituents of a substituted alkyl can include substituted and unsubstituted forms of the groups listed above.

The phrase “aryl alkyl”, as used herein, refers to an alkyl group substituted with an aryl group (e.g., an aromatic or heteroaromatic group).

As used herein, the terms “alkenyl” and “alkynyl” refer to unsaturated aliphatic groups analogous in length and optional substitution to the alkyls groups described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents such as those described above for alkyl groups. The term “aryl” also includes polycyclic ring systems having two or more cyclic rings in which two or more carbons are common to two adjoining rings (the rings are “fused rings”) wherein at least one of the rings is aromatic, e.g., the other cyclic ring(s) can be cycloalkyls, cycloalkenyls, cycloalkynyls, aryls and/or heterocyclyls.

As used herein, the terms “heterocycle” or “heterocyclic group” refer to 3- to 12-membered ring structures, e.g., 3- to 7-membered rings, whose ring structures include one to four heteroatoms. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents such as those described for alkyl groups.

As used herein, the term “heteroatom” as used herein means an atom of any element other than carbon or hydrogen, such as nitrogen, oxygen, sulfur and phosphorous.

### The Present Sensor

The present invention relates to sensors including artificial receptors and methods of using them. In an embodiment, the present invention includes an artificial receptor (e.g., a working artificial receptor) as a component of a receptor system including a ligand permeable interface (e.g., a semipermeable membrane) that isolates the artificial receptor (e.g., a working artificial receptor) from certain components of the surrounding environment. In such a receptor system, the ligand of interest can cross (e.g., diffuse through) the ligand permeable interface and enter a fluid composition in contact with the artificial receptor. The ligand of interest can then bind to the artificial receptor.

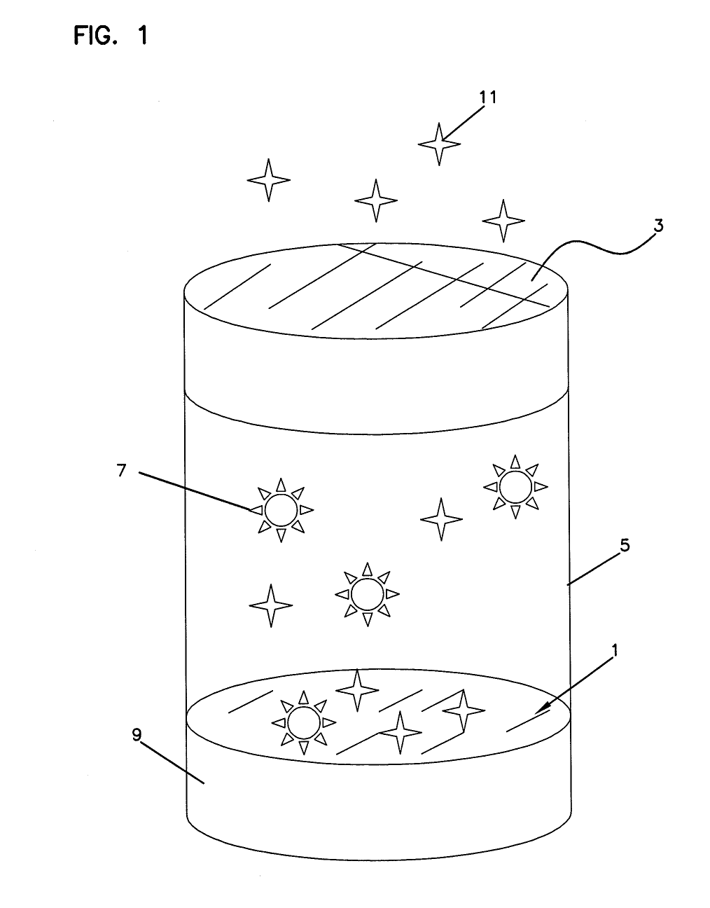

FIG. 1 schematically illustrates an embodiment of the present receptor system including an artificial receptor 1, ligand permeable interface 3, wall 5, and optional features. Wall 5 and ligand permeable interface 3 isolate artificial receptor 1 from its surroundings.

In an embodiment, the present invention includes an artificial receptor and a competitor against a ligand of interest. The competitor is configured or selected to compete with the ligand of interest for binding to the artificial receptor. Similarly, the artificial receptor is configured or selected to bind the ligand of interest and the competitor and for binding of the ligand of interest to compete with binding of the conjugate to the artificial receptor. That is, increasing the concentration of the ligand of interest over a range decreases the amount of competitor bound to the artificial receptor. The competition can result in a significant change in signal due to binding (e.g., significant decrease in signal from binding of competitor to the artificial receptor or significant increase in signal from binding of the ligand of interest to artificial receptor). Such an artificial receptor is referred to herein as a “competitive artificial receptor”.

FIG. 1 schematically illustrates an embodiment of the present invention including artificial receptor 1, competitor 7, and optional features. Competitor 7 and ligand 11 can compete for binding to the artificial receptor 1.

In an embodiment, the present invention includes a competitive artificial receptor as a component of a detector system including a semipermeable membrane that isolates the competitive artificial receptor from certain components of the surrounding environment. This embodiment also includes the competitor described above and a detector operatively coupled to the competitive artificial receptor. The detector produces a signal indicating binding of the competitor and/or the ligand of interest to the artificial receptor. The detector system is configured so that the competitor is retained in the environs of the artificial receptor at least in part by the ligand permeable interface. For example, in such a detector system, the ligand permeable interface, artificial receptor, and another member, e.g., a wall, can define a chamber and the competitor can be in the chamber. The ligand of interest can cross (e.g., diffuse through) the ligand permeable interface and enter fluid in (e.g., isotonic liquid filling) the chamber. The ligand of interest can then bind to the artificial receptor in competition with the competitor. The competition results in a significant change in signal due to binding (e.g., significant decrease in signal from binding of competitor to the artificial receptor or significant increase in signal from binding of the ligand of interest to the artificial receptor).

FIG. 1 schematically illustrates an embodiment of the detector system including artificial receptor 1, ligand permeable interface 3, wall 5, competitor 7, and detector 9. Detector 9 produces a detectable signal in response to binding to the artificial receptor 1. Competitor 7 and ligand 11 can compete for binding to the artificial receptor 1, which can result in the detectable signal or a change in the detectable signal. The detectable signal can indicate a level or concentration of the ligand of interest.

In an embodiment, the invention relates to an artificial receptor system. This system includes a molecular competitor, an artificial receptor, a ligand permeable interface, and a detector. The molecular competitor can competes against binding of a ligand of interest to the artificial receptor. The artificial receptor is configured to bind a ligand of interest and the competitor, binding of the ligand of interest competing with binding of the competitor. The artificial receptor includes a plurality of different building block molecules independently covalently coupled to a solid support. The ligand permeable interface can be permeable to the ligand of interest but retain an effective amount of the competitor. The ligand permeable interface and the artificial receptor can at least partially define a chamber. The competitor and the artificial receptor can be in the chamber. The detector can be operatively coupled to the artificial receptor and configured to produce indicia of presence or concentration of the ligand of interest.

### RFID Detector

In an embodiment, the detector can include a signal processing component that is operatively coupled to a data communication component, preferably enabled for RFID communication although other means of communication are possible. The RFID enabled data communication component includes electronic circuitry adapted for transmitting glucose data signal to a remote transponder. Implantable RFID devices are known, for example, for measuring body temperature of companion animals, for identifying companion animals, and the like. The implantable device can be enclosed in glass or plastic (e.g., the wall can be glass or plastic) with the glucose permeable interface also on a surface of the device. Suitable implantable RFID circuitry can be enclosed in a glass or plastic body comparable in size to a grain or rice. Alternatively, the device can be larger than a grain of rice, but small enough to implant comfortably under the skin of or intramuscularly in a mammal (e.g., a human). The device can be made small enough to be swallowed or implanted using minimally invasive procedures. Smaller in vivo devices can be implanted using a catheter or other injection system and are preferred in the present invention.

### A Glucose Sensor

The present invention relates to a glucose sensor including an artificial receptor and to methods of using it. In an embodiment, the receptor system includes an artificial glucose receptor, a glucose permeable interface (e.g., a semipermeable membrane) that isolates the artificial glucose receptor from certain components of the surrounding environment (e.g., macromolecules in blood or another biological fluid). In this embodiment of the receptor system, glucose, when present, can cross (e.g., diffuse through) the glucose permeable interface, enter liquid in contact with the artificial glucose receptor, and bind to the artificial glucose receptor.

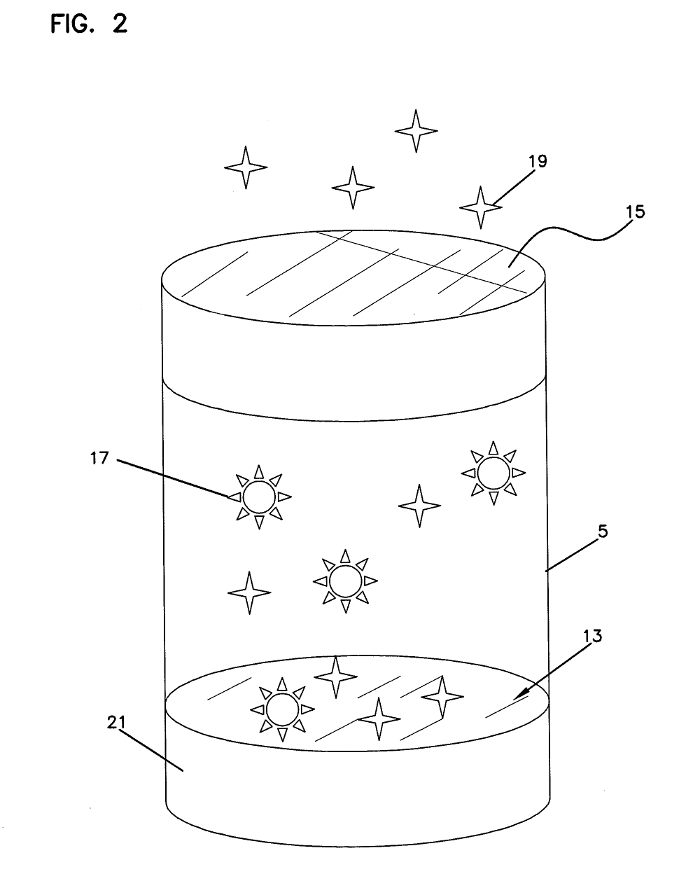

FIG. 2 schematically illustrates an embodiment of the present receptor system including an artificial glucose receptor 13, glucose permeable interface 15, wall 5, and optional features. Wall 5 and glucose permeable interface 15 isolate artificial glucose receptor 13 from its surroundings.

In an embodiment, the present invention includes an artificial glucose receptor and a glucose-competitor. The glucose-competitor is configured or selected to compete with glucose for binding to the artificial receptor. Similarly, the artificial glucose receptor is configured or selected to bind both glucose and the glucose-competitor and for competition between binding of these two moieties. For example, in this embodiment, increasing the concentration of glucose over a physiologically relevant range decreases the amount of glucose-competitor bound to the artificial receptor. Such competition results in a significant change in signal, for example a significant decrease in signal from binding of the glucose-competitor to the artificial glucose receptor or significant increase in signal from binding of glucose to the artificial glucose-receptor.

FIG. 2 schematically illustrates an embodiment of the glucose-competitor system including artificial glucose receptor 13, glucose-competitor 17, and optional features. Glucose-competitor 17 and glucose 19 can compete for binding to the artificial glucose receptor 13.

In an embodiment, the detector system includes an artificial glucose receptor, a glucose permeable interface, a glucose-competitor, and a glucose detector. The glucose permeable interface isolates the artificial glucose receptor from certain components of the surrounding environment. The glucose detector is operatively coupled to the artificial glucose receptor and produces a signal indicating binding of the glucose-competitor and/or glucose to the artificial glucose receptor. This embodiment is configured so that the glucose-competitor is retained in the environs of the artificial glucose receptor at least in part by the glucose permeable interface. For example, the glucose permeable interface, artificial glucose receptor, and another member, e.g., a wall, can define a detector chamber and the glucose-competitor can be in the detector chamber. The glucose can cross (e.g., diffuse through) the glucose permeable interface and enter liquid in the detector chamber. The glucose can then bind to the artificial glucose receptor in competition with the glucose-competitor. The competition results in a significant change in signal due, e.g., to a significant decrease in signal from binding of the glucose-competitor to the artificial receptor or to a significant increase in signal from binding of glucose to the artificial receptor).

FIG. 2 schematically illustrates an embodiment of a glucose detector system including the artificial glucose receptor 13, the glucose permeable interface 15, wall 5, glucose-competitor 17, and glucose signal transponder 21. Glucose signal transponder 21 produces a detectable signal in response to binding to the artificial glucose receptor 13. Glucose-competitor 17 and glucose 19 can compete for binding to the artificial glucose receptor 13, which can result in the detectable signal or a change in the detectable signal. The detectable signal can indicate a level or concentration of glucose 19.

In an embodiment, the artificial glucose receptor and the competitor are configured to provide competition between glucose and the competitor for the artificial glucose receptor at concentrations of glucose that are achieved in the detector system when the detector system is exposed to a physiological concentration of glucose. The detector can be configured provide indicia of a low concentration of glucose, an acceptable concentration of glucose, and a high concentration of glucose.

In operation, the glucose sensor can be implanted in a subject so that it is in fluid communication with a biological fluid that contains a level of glucose indicative of blood glucose levels. For example, the glucose sensor can be implanted in a blood vessel or in a tissue through which blood is circulated, e.g. muscle or skin. Glucose from the bloodstream moves (e.g., diffuses) through the glucose permeable interface and into the detector chamber. Reversible binding of glucose to the artificial receptor is detected by and a signal is produced by the glucose detector. The signal represents glucose concentration in the blood. In an embodiment, the signal can be transformed into a read-out of “LOW-SAFE-HIGH” readings based on the glucose levels in the blood stream.

In an embodiment, the artificial glucose receptor and/or the competitor in the detector system is configured or selected to provide competition of glucose and competitor for the receptor at concentrations of glucose that are achieved in the detector system when the detector system is exposed to physiological (e.g., blood or plasma) concentrations of glucose or is implanted in a tissue that includes glucose. In an embodiment, the artificial glucose receptor and/or the competitor in the detector system is configured or selected so that there is not detectable or only an insignificant level of competition for the receptor between biomolecules (e.g., sugars) other than glucose and the competitor. The artificial glucose receptor and/or the competitor can be selected to provide no detectable or insignificant competition due to physiological concentrations of biomolecules (e.g., sugars) other than glucose, such as fructose and galactose. Put another way, the detector system can be selective or specific for glucose and sensitive to varying ranges of glucose concentrations.

By “selective” is meant that the artificial glucose receptor and/or the competitor is specific for glucose molecules rather than other biomolecules, e.g., sugars such as fructose and galactose. By “sensitive” is meant the affinity of the competitor for the artificial glucose receptor is such that a suitable signal with acceptably low levels of interference is produced. In an embodiment, the artificial glucose receptor and the competitor produce a signal indicative of glucose level in the presence of physiological concentrations of glucose, fructose, and galactose, e.g., 80 to 120 mg/dL glucose, 2-12 mg/dL fructose, and 1.5-90 mg/dL galactose.

Glucose detection by the glucose sensor system can be controlled passively or actively. As used herein, the term “passive control” refers to those embodiments in which glucose detection and quantification is initiated at a particular time by changes in the environment. In an embodiment, changes in glucose level triggers quantification of glucose in the bloodstream by the glucose sensor device. In the passive sensing embodiments, glucose quantification can be triggered by environmental glucose changes, for example, by eating, exercises, sleeping, resting, or other psychosomatic response after placement of the device onto or into the body of a human or other animal. In addition, as used herein the term “active control” refers to those embodiments in which glucose detection and quantification is initiated at a particular time by the application of a stimulus to the device or a portion of the device. In an embodiment, deliberate quantification of glucose levels is performed after a query is made to the glucose sensor. The passive mechanism differs from the active mechanism in that glucose quantification is triggered by a directly applied query rather than an environmental one.

Active glucose sensor microchip devices may be controlled by local microprocessors or remote control. Glucose biosensor information may provide input to the controller to determine the time and type of activation automatically, with human intervention, or a combination thereof.

### RFID Glucose Detector

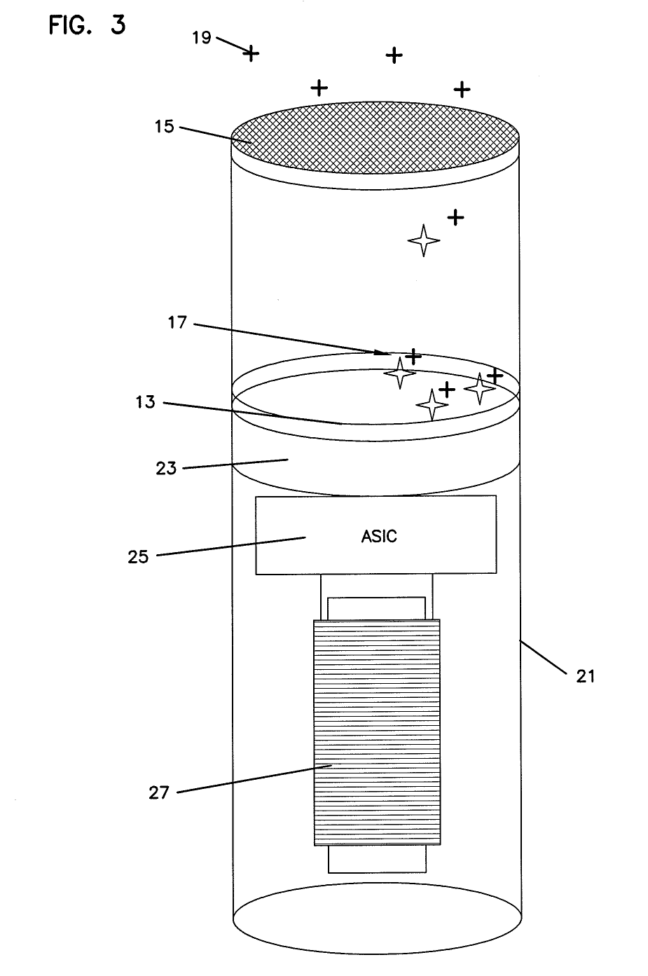

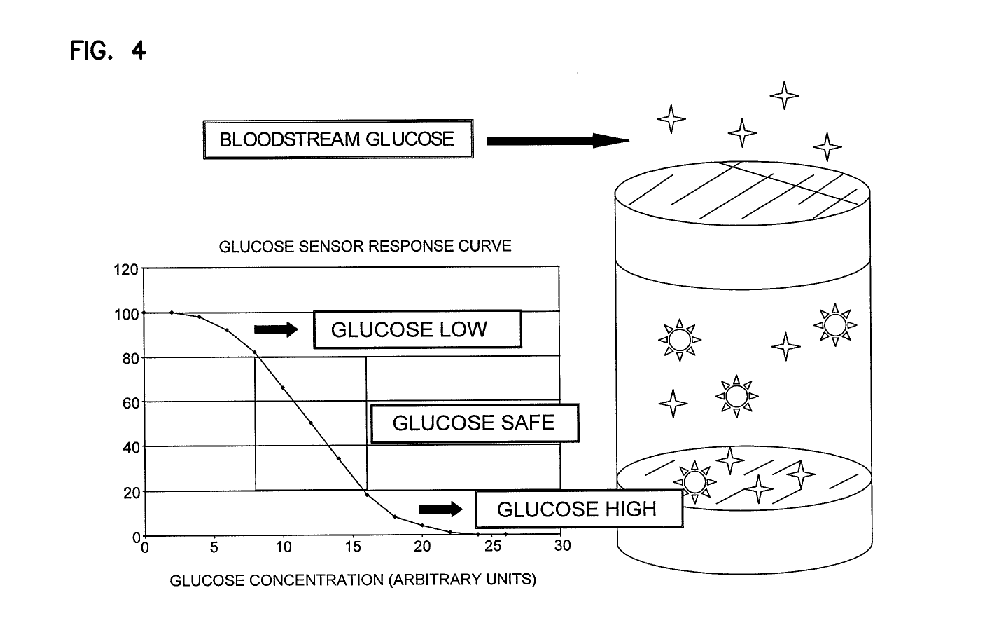

In an embodiment, the glucose detector can include a signal processing component that is operatively coupled to a data communication component, preferably enabled for RFID communication although other means of communication are possible. FIG. 3 schematically illustrates an embodiment of the present glucose detector system that is configured for RFID communication. This embodiment includes the artificial glucose receptor 13, the glucose permeable interface 15, wall 5, glucose-competitor 17, and glucose signal transponder 21.

In the embodiment illustrated in FIG. 3, the glucose signal transponder 21 includes receptor interface 23, circuit 25, and antenna 27. The receptor interface 23 produces a detectable signal in response to binding of glucose 19 or glucose-competitor 17 to the artificial glucose receptor 13. The signal can be, for example, an optical signal, e.g., due to a label on the glucose-competitor, or a mass signal, e.g., due to the difference in mass between glucose 19 and the glucose-competitor 17. For example, the receptor interface 23 can include a wave guide that guides an optical signal from the artificial glucose receptor 13 into the glucose signal transponder 21. For example, the receptor interface 23 can include a microbalance (e.g., quartz crystal microbalance or the like) that detects a mass difference between receptor-bound glucose 19 and receptor-bound glucose-competitor 17 and produces a corresponding signal.

The receptor interface 23 is operatively coupled to the artificial glucose receptor 13 and circuit 25. Circuit 25 is operatively coupled to antenna 27. Circuit 25 and antenna 27 are configured for RFID communication. The binding of glucose 19 to the artificial glucose receptor 13 produces a signal that represents the glucose concentration. The signal is processed by the circuit 25 to provide a signal that can be transmitted in response to power taken in by antenna 27. The circuit 25 transmits the signal through the antenna 27 configured to wirelessly transmit the data to a remote transponder or computer system that reports the glucose concentration based on the signal.

### Competitor

The competitor is a molecule competes with the ligand of interest for binding to the artificial receptor and that is retained in the presence of the artificial receptor by the ligand permeable interface. In an embodiment, the competitor is of a molecular weight large enough to be retained by a porous structure (e.g., a semipermeable membrane) that discriminates on the basis of size. The ligand of interest, then, is small enough to pass through the porous structure of the ligand permeable interface. In an embodiment, the competitor is a conjugate of a macromolecule and an analog of the ligand of interest, or even a conjugate with the ligand itself. As used herein, the term “conjugate” refers to a small molecule covalently coupled to a macromolecule. Thus, the competitor can include an analog of a ligand of interest covalently coupled to a macromolecule.

Suitable macromolecules include one or more reactive functional groups to which the analog of the ligand of interest or the ligand itself can be bound via covalent or noncovalent interactions. In certain embodiments, the macromolecule has one or more of: control of the type and display of surface recognition elements; a narrow molecular weight distribution; terminal groups capable of being functionalized; a high degree of molecular uniformity; size and shape that enables suitable (e.g., maximum) conjugation with the analog of the ligand or interest or the ligand of interest; solubility; and/or availability. Suitable macromolecules include a protein, a polynucleotide, a polysaccharide, another natural polymer, a synthetic polymer, a dendrimer, a combination thereof, or a mixture thereof. In an embodiment, the competitor is a conjugate of a dendrimer and an analog of the ligand of interest or the ligand of interest.

The analog of the ligand of interest can be a molecule with sufficient structural similarity to compete with the ligand of interest of interest for binding to the artificial receptor. When the ligand of interest is glucose, the analog of the ligand of interest can be any glucose analogue that competes with glucose for binding to the artificial glucose receptor. Suitable glucose analogs include galactose, fucose, mannose, glucosamine, galactosamine, and glucose-ITC.

The competitor can also include a detectable label, such as a fluorophone. Suitable detectable labels include perylene dyes, benzoxanthenes, Alexa Fluor 647, Alexa Fluor-594, Alexa Fluor 488 Dye, Alexa Fluor 500 and Alexa Fluor 514 Dyes, Alexa Fluor 532, Alexa Fluor 546, Alexa Fluor 555, Alexa Fluor 568, Alexa Fluor 594 and Alexa Fluor 610 Dyes, Alexa Fluor 633, Alexa Fluor 635, Alexa Fluor 647, Alexa Fluor 660, Alexa Fluor 680, Alexa Fluor 700 and Alexa Fluor 750 Dyes, Alexa Fluor 350 Dye, Alexa Fluor 405 Dye, Alexa Fluor 430 Dye or alternatively inorganic compounds, for example zinc sulfide. Suitable detectable labels include those that can be detected by chemical, mechanical, optical, electrical, electronic, ionic, or mass spec means.

In an embodiment, the competitor includes the macromolecule that is not conjugated to an analog of the ligand of interest or the ligand of interest. In an embodiment, the competitor is the macromolecule.

### Dendrimer

A dendrimer is a spheroid or globular nanostructure configured to carry another molecule encapsulated in its interior void space or attached to its surface. Size, shape, and reactivity are generally determined by generation (shells) and chemical composition of the core, interior branching, and surface functionalities. Dendrimers contain a core to which numerous surface groups are attached covalently. Surface groups can be cationic, anionic, neutral, or hydrophobic.

Dendrimers are constructed through a set of repeating chemical synthesis procedures that build up from the molecular level to the nanoscale region under conditions that are easily performed in a standard organic chemistry laboratory. The dendrimer diameter increases linearly whereas the number of surface groups increases geometrically. Dendrimers are uniform with low polydispersities, and are commonly created with dimensions incrementally grown in approximately nanometer steps from 1 to over 10 nm. Each subsequent growth step represents a new “generation” of polymer with a larger molecular diameter, twice the number of reactive surface sites, and approximately double the molecular weight of the preceding generation.

Dendrimers are by nature of their synthesis, heterodisperse in their structure. This results in a reduced and varied number of reactive surface groups within a batch, due to deletion or dimerization of dendrimer “arms” during synthesis. In order to generate a more structurally homogenous core carrier with respect to the molecular weight distribution for the glucose receptor environment, fractionation by conventional means, such as semi-prep scale HPLC method or the like can be undertaken. Fractionation of dendrimer carriers includes placing a quantity of dendrimer material in the HPLC column and selecting the fraction with the narrowest band that represents the most narrow molecular weight distribution for additional processing. Dendrimers can be attached to support surfaces like glass, gold, silica, semi-permeable membranes and plastics.

Dendrimers can be conjugated to a ligand or ligand analog using standard reactions. For example, when carboxylic acid-terminated dendrimers are used, surface carboxylic acids are activated prior to coupling with ligand or ligand analog. Amine-terminated dendrimers cores do not require surface activation prior to coupling to many ligands or ligand analogs, for example, glucose analogues.

Suitable dendrimers include dendrimers of poly(amidoamine) on an ethylenediamine core, known as PAMAM dendrimers and commercially available from DENDRITECH®, Inc., Midland, Mich. Such dendrimers can be synthesized to include “generations” of dendritic growth, for example 0, 1, 2, 3, 4, 4.5 or 5 generations. PAMAM dendrimers are generally characterized as “dense star” polymers. Unlike classical polymers, PAMAM dendrimers have a high degree of molecular uniformity, narrow molecular weight distribution, specific size and shape characteristics, and a highly-functionalized terminal surface. Table I lists properties for several generations of dendrimers of poly(amidoamine) on an ethylenediamine core.

TABLE 1

| | Molecular | Measured | Surface |
|---|---|---|---|
| Generation | Weight | Diameter (Å) | Groups |
| 0 | 517 | 15 | 4 |
| 1 | 1,430 | 22 | 8 |
| 2 | 3,256 | 29 | 16 |
| 3 | 6,909 | 36 | 32 |
| 4 | 14,215 | 45 | 64 |
| 5 | 28,826 | 54 | 128 |
| 6 | 58,048 | 67 | 256 |
| 7 | 116,493 | 81 | 512 |
| 8 | 233,383 | 97 | 1024 |
| 9 | 467,162 | 114 | 2048 |
| 10 | 934,720 | 135 | 4096 |

Suitable dendrimers include generation 3.0 dendrimers of poly(amidoamine) on an ethylenediamine core. Generation 3.0 can theoretically be coupled to 32 receptor ligands per core molecule. In experiments, generation 3.0 demonstrated a 99.3% coupling efficiency as an average of 31.8 glucose analogue molecules were coupled to the carrier molecule. In contrast, generation 5.0 can theoretically hold up to 128 receptor ligands per core molecule. Coupling experiments show that an average of 100.5 glucose analogue molecules could be added which is 78.5% efficiency.

Other suitable dendrimers include amine-terminated PAMAM dendrimers available from DENDRITECH®, Inc., Midland, Mich. Amine-terminated dendrimers do not have to undergo an activation step prior to coupling to ligand or ligand analog. In addition, conjugation can employ more robust and reproducible chemistry, such as the amine-isothiocyanate (ITC) reaction.

In an embodiment, a PAMAM dendrimer can be conjugated to ligand or ligand analog according to the following general procedure. Surface groups of the dendrimer molecule can be activated or labeled, functionalized and/or capped via a series of parallel chemical reactions that involves acetylation, labeling with a fluorescent agent and/or acetylation that adds on a carboxylic group. By “activation” is meant introduction of a labile group onto the dendrimer molecule that can be easily cleaved or removed in subsequent steps so that addition of other functional moieties is readily accomplished. In one embodiment, the dendrimer molecule is neutralized under acidic conditions followed by addition of 1-ethyl-3-(3-dimethylaminopropyl) carbodiimide (EDC) in a DMF/H20 mixture. Next, N-hydroxysuccinimide (NHS) ester is added to form the activated materials that are carried forward without purification. To the activation reaction is added a fluorescent label, such as Alexa-647 followed by the glucose derivative or analog, such as an amine-bearing glucose analogue to form a functionalized competitor.

In another embodiment, a dendrimer molecule with amine-bearing surface groups is reacted with sugar analogues without having to undergo an activation step prior to coupling. The glucose analogues are added through a robust and reproducible coupling chemistry like an amine-isothiocyanate (ITC) reaction to form functionalized competitor agents that bind competitively to glucose. In a third embodiment, dendrimer cores are acetylated, labeled with a fluorescent dye and/or coupled to a glucose analogue in order to form functionalized competitor agents suitable for binding glucose in the present invention.

Any excess solvent can be removed via lyophilization or the like. Additional purification steps can be accomplished via size exclusion chromatography, dialysis, HPLC or the like. The purification scheme that is selected can be based on the amount of material to be purified and the degree of contamination. In one embodiment, the dendrimer molecule is fractionated prior to activation and/or functionalization to minimize variable competitor agent performance and obtain dendrimer starting materials that has a narrower molecular weight distribution.

The competitor can be characterized by one or more analytical tests to confirm purity, to study binding interactions, to estimate the number of ligand or ligand analog molecules that have been added, to confirm structural changes, or the like. In an embodiment, HPLC is used to confirm purity of competitor candidates prior to and after functionalization. In an embodiment, mass spectrometry is used to estimate the number of glucose residues that have been added to the dendrimer core. In an embodiment, NMR is used to confirm any structural changes to the dendrimer cores as a result of addition glucose analogues.

Reagents and a reaction scheme suitable for producing a competitor of the present invention are illustrated below in Schemes A and B.

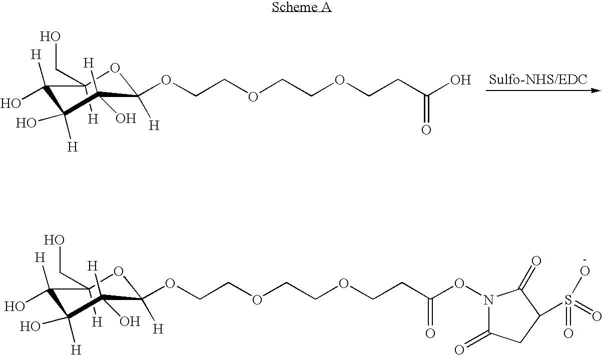

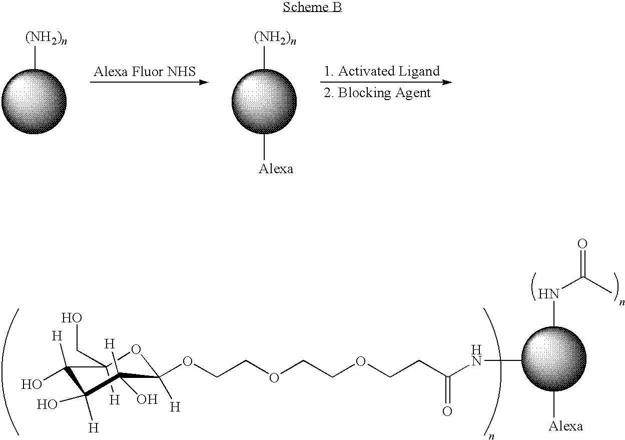

### Ligand Permeable Interface

The ligand permeable interface separates the artificial receptor from the surroundings external to the sensor, e.g., body fluid and tissue. The ligand permeable interface is configured or selected to allow the ligand of interest to enter the sensor and to contact the artificial receptor. The ligand permeable interface can be compatible with body fluid and tissue, it can be biocompatible. The ligand permeable interface prevents the artificial receptor from being in direct contact with living tissue and biological fluid and undesired reaction with them.

The ligand permeable interface can be a semipermeable membrane. The semipermeable membrane can be selected, for example, to allow a molecule the size of the ligand of interest to enter the fluid that contacts the receptor and to exclude larger molecules. In an embodiment, the semipermeable membrane is permeable to the ligand of interest but retains an effective amount of the competitor.

In an embodiment, the semipermeable membrane and the artificial receptor (e.g. its support) at least partially define a chamber. The competitor and the artificial receptor can be in the chamber. By partially define is meant that the semipermeable membrane and the artificial can be configured to form the chamber or that another member can also participate in defining the chamber. For example the chamber can be defined by the semipermeable membrane, the artificial receptor (e.g., its support), and a wall or walls or a capsule or other structure.

The semipermeable membrane can be selected to exclude molecules with particular characteristics (e.g., more than a predefined degree of charge or lipophilicity) but to be porous to molecules of other characteristics (e.g., less than a predefined degree of charge or lipophilicity). The semipermeable membrane can be permeable to a ligand, such as glucose, but impermeable to a competitor, such as a dendrimer-sugar conjugate. The semipermeable membrane can be impermeable to other components of biological fluids, such as proteins, cells, polysaccharides, and the like. The semipermeable membrane, artificial receptor, and a wall can define a chamber.

The ligand permeable interface (e.g., semipermeable membrane) can be selected to exclude molecules with a molecular weight above a predetermined exclusion limit. For example, the exclusion limit can be smaller than the molecular weight of the competitor and proteins in the biological fluid, but larger than the molecular weight of the ligand of interest, e.g., glucose.

The ligand permeable interface (e.g., semipermeable membrane) can be made of a material that resists degradation by biological fluids or tissue. Suitable materials include glass, ceramic, metal, synthetic polymer (persistent or biodegradable), and biopolymer (persistent or biodegradable). The interface can be formed of only one material or can be a composite or multi-laminate material, e.g., several layers of the same or different interface materials that are bonded together. Composite or multi-laminate substrates can include any number of layers of silicon, glasses, ceramics, semiconductors, metals, polymers.

Representative synthetic, non-degradable polymers include poly(ethers) such as poly(ethylene oxide), poly(ethylene glycol), and poly(tetramethylene oxide); vinyl polymers; poly(acrylates) and poly(methacrylates) such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose; and various cellulose acetates; poly(siloxanes); and any chemical derivatives thereof (substitutions, additions of chemical groups, for example, alkyl, alkylene, hydroxylations, oxidations, and other modifications routinely made by those skilled in the art), copolymers and mixtures thereof. In an embodiment, the semipermeable membrane is composed of a biocompatible polymer including polysulfone, such as a mixture of hydrophilic polymer(s) and hydrophobic polysulfone polymer. Representative synthetic, biodegradable polymers include poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), copolymers and mixtures thereof.

The ligand permeable interface can be a hollow fiber membrane. Such as hollow fiber membrane can be made from a suitable biocompatible material and have a molecular weight cut-off (MWCO) or pore size that allows for exclusion of protein and cellular components found in the blood stream and permeation of the ligand of interest (e.g., glucose).

### Wall and Capsule

In an embodiment, the exterior of the sensor, with the exception of the ligand permeable interface is made of a material that is biocompatible and impermeable. Suitable materials include glass and many plastics. The wall of the sensor can be made of this material. The exterior of the sensor, with the exception of the ligand permeable interface is referred to herein as the capsule. The capsule can define an aperture that is sealed with the ligand permeable interface. This aperture can be on the surface of the capsule or in a channel formed by the capsule. The wall and capsule are composed of a material that will isolate the artificial receptor, detector, and circuitry from biochemical interference. The wall and capsule can be composed of material or materials that resist the aggressive environment present in the body. The wall and capsule can be compatible with both the living tissue and with other materials used to construct the device. Non-biocompatible materials can be encapsulated in or coated by a biocompatible material, such as poly(ethylene glycol) or polytetrafluoroethylene-like materials.

### Detector

The detector is operatively coupled to the competitive artificial receptor and produces a signal indicating or indicia of binding of the competitor and/or the ligand of interest to the artificial receptor. Upon binding to the artificial receptor, the detector produces the signal or indicia due to this binding. For example, the signal or indicia can be produced in response to a significant decrease in binding of competitor to the artificial receptor or from a significant increase in binding of the ligand of interest to the artificial receptor. In an embodiment, the detector is operatively coupled to the artificial receptor and configured to, in response to binding of the competitor or the ligand of interest to the artificial receptor, produce indicia of presence or concentration of the ligand of interest.

The indicia can be a detectable signal. The indicia can be a value that is stored in memory and/or transmitted to a receiver. In an embodiment, the detector produces an electrical signal in response to binding and that corresponds to the level of the ligand of interest. In an embodiment, the detector detects an optical signal from binding and produces an electrical signal that corresponds to the optical signal. In an embodiment, the detector detects mass due to binding the conjugate and produce an electrical signal that corresponds to the mass. The electrical signal can provide an indicia that can be, for example, stored in memory and/or transmitted to a receiver.

In an embodiment, the detector includes an antenna and an integrated circuit. The integrated circuit can be configured to store the indicia, receive a first radio signal, and transmit a second radio signal. The first radio signal can provide power to activate the integrated circuit to transmit the second radio signal. The second radio signal includes the indicia.

The detector can include any of a variety of components or circuitry for measuring or analyzing the presence, absence, or change in a chemical or ionic species, electromagnetic or thermal energy (e.g., light), or one or more physical properties (e.g., pH, pressure) at a site. The detector can include any of a variety of components or circuitry for detecting and producing signals, for example, a microprocessor or controller. The detector can transmit the signal or information derived from the signal to a remote controller, to another local controller, or both. In an embodiment, the microprocessor or controller can relay or record information on the level of the ligand (e.g., glucose) at a site in a subject.

In an embodiment, the detector includes an optical sensor system. The optical sensor system can include a waveguide, a detection system operatively coupled to the waveguide, and an artificial receptor that binds the ligand (e.g., glucose). The waveguide can be operatively configured with respect to the artificial receptor such that the waveguide can receive an optical signal (e.g., fluorescence, luminescence, or absorbance) from the artificial receptor.

In an embodiment, the detector includes an electrochemical sensor system. An electrochemical sensing system can include a transducer (e.g., an electrode or CHEMFET), a detection system operatively coupled to the transducer, and an artificial receptor that binds the ligand of interest (e.g., glucose). The transducer can be operatively configured with respect to the working artificial receptor such that the transducer can detect changes in electrical charge, potential, or current (e.g., conductance, capacitance, or impedance) from the working artificial receptor. In an embodiment, the transducer includes at least one electrode. An electrochemical sensing system can include a working electrode, a reference electrode, and an artificial receptor. In an embodiment, the artificial receptor can be coupled to the working electrode. In an embodiment, the artificial receptor can be coupled to a membrane that is configured between the working electrode and the reference electrode. In an embodiment, the working electrode and reference electrode can be conventional electrodes. In an embodiment, the working electrode and reference electrode can be a source and a drain of a field effect transistor.

The artificial receptor can be supported in the detector or sensor system in a variety of configurations in or on a variety of support materials. To accommodate various sensing systems, the present artificial receptors can be configured in an environment that conducts electrical currents, light, and/or other electromagnetic radiation.

The detector can include a communication system. For example, a glucose sensor employing the present artificial receptors can be coupled to a communications network using wired or wireless technology. FIG. 3 schematically illustrates a system employing RFID for communication. The detector can provide data (e.g., glucose level) to a communication system that can be coupled to the internet. A processing system that is also coupled to the communications network can monitor one or more signals from one or more glucose sensors. In an embodiment of a responsive system, corrective action can be taken as necessary in response to signals received from a sensor.

Another embodiment of a communication system includes optical communication, where the receiver is in the form of a photocell, photodiode, and/or phototransistor, and where the transmitter a light-emitting diode (LED) or laser. For telemetry through soft tissue of the body, acoustic (i.e. sonic) energy, such as ultrasound energy, may be used as a means of communication.

In an embodiment, the detector includes a receiver that accepts commands and data from a remote controller, and may be used to request status information about the state of the system or an event log, or to reprogram the controller operating system (e.g., the internal firmware). In an embodiment in which the microchip device is implanted in a human or animal, the remote controller can include a means of display and/or actuation that can be used by the physician or patient to operate and monitor the microchip device.

### Detector for Glucose Sensor

In a preferred embodiment, an implantable glucose sensor includes an artificial glucose receptor operatively coupled to a detector through an interface located between the artificial glucose receptor and the detector. The signal transduction interface is operatively coupled to an electrical or electronic circuit that converts the signal into a form that can be transmitted to a remote device capable of converting the signal into glucose concentration. The artificial glucose receptor may be immobilized on the interface or the signal transduction component or detector.

Changes in the glucose concentration can be monitored, according to the present invention, by continuously detecting binding to the artificial glucose receptor, e.g., binding of the competitor to the receptor. Specifically, displacement of the competitor by glucose induces a physicochemical change, such as, for example, a photochemical change, a change in light absorption, light emission, light scattering, or light polarization; or an electrochemical or piezoelectric change. In an embodiment, the mass bound to the receptor is converted into a detectable signal in the form of an optical, electrochemical or piezoelectric response. The optical, electrochemical or piezoelectric signal is transmitted to a circuit that correlates the signal to a glucose concentration. In another embodiment, binding of glucose causes displacement of fluorescently labeled competitor to result in a decrease in fluorescence emission. The change in fluorescence emission relative to a preselected state is detected, transmitted to a circuit that correlates the change in fluorescence emission to a specific glucose concentration.

When the glucose-selective binding environment is constructed on a surface that is integral with the signal detector, such as a gold surface or multiplexed gold chip that is capable of binding glucose and the competitor agent, glucose binding can be detected via a signal that results from the addition or subtraction of mass from the surface of the signal detector.

### Power Supply

The detector can include a power supply. The power supply can be a precharged power source (which contain all of the power required for operation over the life of the microchip device), a source that can be periodically recharged, or an on-demand power source. The rechargeable power source (i.e. the rechargeable power storage unit) can store power, but advantageously need not store all of the power required for the operating life of the microchip. The rechargeable power source and on-demand power sources can both be included in a single microchip device, as it is common for a system having an on-demand power source to include a power storage unit, such as a capacitor or battery. Systems and techniques for on-demand power by wireless transmission, which can be adapted for use with the present sensor, are disclosed, for example, in U.S. Pat. No. 6,047,214 to Mueller, et al.; U.S. Pat. No. 5,841,122 to Kirchhoff; U.S. Pat. No. 5,807,397 to Barreras; and U.S. Pat. No. 5,324,316.

In an RFID embodiment, the sensor includes a transducer for receiving energy wirelessly transmitted to the device, circuitry for directing or converting the received power into a form that can be used or stored, and if stored, a storage device, such as a rechargeable battery or capacitor.

The present sensor can be configured to receive power by a variety of means. For example, the present sensor can be configured to receive power from an electromagnetic (EM) energy source, or an acoustic (i.e. sonic) energy or other mechanical energy source. Electromagnetic energy refers to the full spectral range from x-ray to infrared. Representative examples of useful EM energy forms include radio frequency signals and laser light. A representative example of a useful form of acoustic energy is ultrasound. In various embodiments, the rechargeable power storage unit can include, for example, a coil for the receipt of electromagnetic energy, or a means for transducing other types of energy, such as a photocell, a hydrophone, or a combination thereof. Additional components may include a means of power conversion such as a rectifier, a power storage unit such as a battery or capacitor, and an electric potential/current controller (i.e. potentiostat/galvanostat).

The present sensor can include a component to convert mechanical or chemical energy from the body of the human or animal into power (i.e. energy) which can be used to power the detector. For example, components comprising accelerometers and gyroscopes, can be used to convert motion of a body into electrical energy. Similarly, an implanted transducer can convert heartbeats into useful energy, as currently is done with some pacemaker designs. See, e.g., U.S. Pat. No. 5,713,954. In another embodiment, power is generated/converted from a chemical energy source. For example, the microchip can include a biofuel cell which generates the power by chemically reacting a molecule present in the body. Examples of these fuel cells are described for example in Palmore & Whitesides, “Microbial and Enzymatic Biofuel Cells,” Enzymatic Conversion of Biomass for Fuel Production, ACS Symposium Series 566:271-90 (1994); Kano & Ikeda, “Fundamentals and practices of mediated bioelectrocatalysis,” Analytical Sci., 16(10):1013-21 (2000); and Wilkenson, Autonomous Robots, 9(2): 99-111 (2000). In an embodiment, the implanted device would have an immobilized enzyme which would react with a biological molecule to cause electron transfer, thereby causing an electric current to flow. Possible useful biological molecules include triphosphates, such as ATP, and carbohydrates, such as sugars, like fructose.

Many of these components (except for the external energy transmission source) may be fabricated on the microchip (“on-chip” components) using known MEMS fabrication techniques, which are described, for example, in Madou, Fundamental of Microfabrication (CRC Press, 1997) or using known microelectronics processing techniques, which are described, for example, in Wolf & Tauber, Silicon Processing for the VLSI Era (Lattice Press, 1986). Each of these components (except the external energy transmission source) also may exist as discrete, “off the shelf” microelectronic components that can be connected through the use of hybrid electronic packaging or multi-chip modules (MCMs).

The particular power needs of the present sensor will depend on the application for and the specific design of the device. Examples of design factors include the size requirements and anticipated operating life of the device. The particular devices and techniques for transmitting power will likely depend on the selected sites for the sensor and remote transmitter.

### Methods Employing the Present Sensors

In an embodiment, the present invention includes a method of determining a level of a ligand of interest. The method can include implanting in a subject a detector system and retrieving from the detector system indicia of presence or concentration of the ligand of interest.

The method can also include, for example, when the ligand of interest is glucose, implanting the detector system in a subject so that it is in fluid communication with a biological fluid that contains a level of glucose indicative of blood glucose levels. In an embodiment, the artificial glucose receptor and the competitor are configured to provide competition of glucose and competitor for the artificial glucose receptor at concentrations of glucose that are achieved in the detector system when the detector system is exposed to a physiological concentration of glucose. The method can employ a detector configured to provide indicia of a low concentration of glucose, an acceptable concentration of glucose, and a high concentration of glucose.

In an embodiment, the method employs a reader for generating and/or detecting a signal from the detector. In such an embodiment, retrieving the indicia can include disposing a reader proximal the implanted detector system. The reader can be configured to transmit the first radio signal; to receive the second radio signal; and to display information regarding the presence or concentration of the ligand of interest.

### Artificial Receptors

The present receptors include heterogeneous and immobilized combinations of building block molecules. In certain embodiments, the present receptors include combinations of 2, 3, 4, or 5 distinct building block molecules immobilized near one another on a support. A candidate artificial receptor, a lead artificial receptor, or a working artificial receptor includes combination of building blocks immobilized on, for example, a support (e.g., a solid support). The building blocks can be immobilized through any of a variety of interactions, such as covalent, electrostatic, or hydrophobic interactions. For example, the building block and support or lawn can each include one or more functional groups or moieties that can form covalent, electrostatic, hydrogen bonding, van der Waals, or like interactions. In an embodiment, the artificial receptor includes a plurality of different building block molecules independently covalently coupled to a solid support in a region. The region can be a contiguous portion of the surface of the solid support with the different building block molecules distributed randomly throughout the contiguous region.

One or more lead artificial receptors can be developed from a plurality of candidate artificial receptors. In an embodiment, a lead artificial receptor includes a combination of building blocks and binds detectable quantities of ligand of interest upon exposure to, for example, several picomoles of ligand of interest at a concentration of 1, 0.1, or 0.01 μg/ml, or at 1, 0.1, or 0.01 ng/ml ligand of interest; at a concentration of 0.01 μg/ml, or at 1, 0.1, or 0.01 ng/ml ligand of interest; or a concentration of 1, 0.1, or 0.01 ng/ml ligand of interest.

One or more working artificial receptors can be developed from one or more lead artificial receptors. In an embodiment, a working artificial receptor includes a combination of building blocks and binds categorizing or identifying quantities of ligand of interest upon exposure to, for example, several picomoles of ligand of interest at a concentration of 100, 10, 1, 0.1, 0.01, or 0.001 ng/ml ligand of interest; at a concentration of 10, 1, 0.1, 0.01, or 0.001 ng/ml ligand of interest; or a concentration of 1, 0.1, 0.01, or 0.001 ng/ml ligand of interest.

### Building Blocks

The present invention relates to building blocks for making or forming candidate artificial receptors. Building blocks can be designed, made, and selected to provide a variety of structural characteristics among a small number of compounds. A building block can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A building block can be bulky or it can be small.

A building block can be visualized as including several components, such as one or more frameworks, one or more linkers, and/or one or more recognition elements. The framework can be covalently coupled to each of the other building block components. The linker can be covalently coupled to the framework. The linker can be coupled to a support through one or more of covalent, electrostatic, hydrogen bonding, van der Waals, or like interactions. The recognition element can be covalently coupled to the framework. In an embodiment, a building block includes a framework, a linker, and a recognition element. In an embodiment, a building block includes a framework, a linker, and two recognition elements.

A description of general and specific features and functions of a variety of building blocks and their synthesis can be found in copending U.S. patent application Ser. Nos. 10/244,727, filed Sep. 16, 2002, 10/813,568, filed Mar. 29, 2004, and Application No. PCT/US03/05328, filed Feb. 19, 2003, each entitled “ARTIFICIAL RECEPTORS, BUILDING BLOCKS, AND METHODS”; U.S. patent application Ser. Nos. 10/812,850 and 10/813,612, and application No. PCT/US2004/009649, each filed Mar. 29, 2004 and each entitled “ARTIFICIAL RECEPTORS INCLUDING REVERSIBLY IMMOBILIZED BUILDING BLOCKS, THE BUILDING BLOCKS, AND METHODS”; and U.S. Provisional Patent Application Nos. 60/499,965, filed Sep. 3, 2003, and 60/526,699, filed Dec. 2, 2003, each entitled BUILDING BLOCKS FOR ARTIFICIAL RECEPTORS; the disclosures of which are incorporated herein by reference. These patent documents include, in particular, a detailed written description of: function, structure, and configuration of building blocks, framework moieties, recognition elements, synthesis of building blocks, specific embodiments of building blocks, specific embodiments of recognition elements, and sets of building blocks.

### Framework

The framework can be selected for functional groups that provide for coupling to the recognition moiety and for coupling to or being the linking moiety. The framework can interact with the ligand as part of the artificial receptor. In an embodiment, the framework includes multiple reaction sites with orthogonal and reliable functional groups and with controlled stereochemistry. Suitable functional groups with orthogonal and reliable chemistries include, for example, carboxyl, amine, hydroxyl, phenol, carbonyl, and thiol groups, which can be individually protected, deprotected, and derivatized. In an embodiment, the framework has two, three, or four functional groups with orthogonal and reliable chemistries. In an embodiment, the framework has three functional groups. In such an embodiment, the three functional groups can be independently selected, for example, from carboxyl, amine, hydroxyl, phenol, carbonyl, or thiol group. The framework can include alkyl, substituted alkyl, cycloalkyl, heterocyclic, substituted heterocyclic, aryl alkyl, aryl, heteroaryl, heteroaryl alkyl, and like moieties.

A general structure for a framework with three functional groups can be represented by Formula 1a:

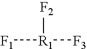

A general structure for a framework with four functional groups can be represented by Formula 1b:

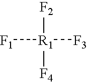

In these general structures: R₁ can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group; and F₁, F₂, F₃, or F₄ can independently be a carboxyl, amine, hydroxyl, phenol, carbonyl, or thiol group. F₁, F₂, F₃, or F₄ can independently be a 1-12, a 1-6, a 1-4 carbon alkyl, substituted alkyl, cycloalkyl, heterocyclic, substituted heterocyclic, aryl alkyl, aryl, heteroaryl, heteroaryl alkyl, or inorganic group substituted with carboxyl, amine, hydroxyl, phenol, carbonyl, or thiol group. F₃ and/or F₄ can be absent.

A variety of compounds fit the formulas and text describing the framework including amino acids, and naturally occurring or synthetic compounds including, for example, oxygen and sulfur functional groups. The compounds can be racemic, optically active, or achiral. For example, the compounds can be natural or synthetic amino acids, α-hydroxy acids, thioic acids, and the like.

Suitable molecules for use as a framework include a natural or synthetic amino acid, particularly an amino acid with a functional group (e.g., third functional group) on its side chain. Amino acids include carboxyl and amine functional groups. The side chain functional group can include, for natural amino acids, an amine (e.g., alkyl amine, heteroaryl amine), hydroxyl, phenol, carboxyl, thiol, thioether, or amidino group. Natural amino acids suitable for use as frameworks include, for example, serine, threonine, tyrosine, aspartic acid, glutamic acid, asparagine, glutamine, cysteine, lysine, arginine, histidine. Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups. Suitable synthetic amino acids include β-amino acids and homo or β analogs of natural amino acids. In an embodiment, the framework amino acid can be serine, threonine, or tyrosine, e.g., serine or tyrosine, e.g., tyrosine.

Although not limiting to the present invention, a framework amino acid, such as serine, threonine, or tyrosine, with a linker and two recognition elements can be visualized with one of the recognition elements in a pendant orientation and the other in an equatorial orientation, relative to the extended carbon chain of the framework.

All of the naturally occurring and many synthetic amino acids are commercially available. Further, forms of these amino acids derivatized or protected to be suitable for reactions for coupling to recognition element(s) and/or linkers can be purchased or made by known methods (see, e.g., Green, T W; Wuts, P G M (1999), Protective Groups in Organic Synthesis Third Edition, Wiley-Interscience, New York, 779 pp.; Bodanszky, M.; Bodanszky, A. (1994), The Practice of Peptide Synthesis Second Edition, Springer-Verlag, New York, 217 pp.).

### Recognition Element

The recognition element can be selected to provide one or more structural characteristics to the building block. The recognition element can interact with the ligand as part of the artificial receptor. For example, the recognition element can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A recognition element can be a small group or it can be bulky.

In an embodiment the recognition element can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group. The recognition element can be substituted with a group that includes or imparts positive charge, negative charge, acid, base, electron acceptor, electron donor, hydrogen bond donor, hydrogen bond acceptor, free electron pair, π electrons, charge polarization, hydrophilicity, hydrophobicity, and the like.

Recognition elements with a positive charge (e.g., at neutral pH in aqueous compositions) include amines, quaternary ammonium moieties, sulfonium, phosphonium, ferrocene, and the like. Suitable amines include alkyl amines, alkyl diamines, heteroalkyl amines, aryl amines, heteroaryl amines, aryl alkyl amines, pyridines, heterocyclic amines (saturated or unsaturated, the nitrogen in the ring or not), amidines, hydrazines, and the like. Alkyl amines generally have 1 to 12 carbons, e.g., 1-8, and rings can have 3-12 carbons, e.g., 3-8. Suitable alkyl amines include that of formula B9. Suitable heterocyclic or alkyl heterocyclic amines include that of formula A9. Suitable pyridines include those of formulas A5 and B5. Any of the amines can be employed as a quaternary ammonium compound. Additional suitable quaternary ammonium moieties include trimethyl alkyl quaternary ammonium moieties, dimethyl ethyl alkyl quaternary ammonium moieties, dimethyl alkyl quaternary ammonium moieties, aryl alkyl quaternary ammonium moieties, pyridinium quaternary ammonium moieties, and the like.

Recognition elements with a negative charge (e.g., at neutral pH in aqueous compositions) include carboxylates, phenols substituted with strongly electron withdrawing groups (e.g., substituted tetrachlorophenols), phosphates, phosphonates, phosphinates, sulphates, sulphonates, thiocarboxylates, and hydroxamic acids. Suitable carboxylates include alkyl carboxylates, aryl carboxylates, and aryl alkyl carboxylates. Suitable phosphates include phosphate mono-, di-, and tri-esters, and phosphate mono-, di-, and tri-amides. Suitable phosphonates include phosphonate mono- and di-esters, and phosphonate mono- and di-amides (e.g., phosphonamides). Suitable phosphinates include phosphinate esters and amides.

Recognition elements with a negative charge and a positive charge (at neutral pH in aqueous compositions) include sulfoxides, betaines, and amine oxides.

Acidic recognition elements can include carboxylates, phosphates, sulphates, and phenols. Suitable acidic carboxylates include thiocarboxylates. Suitable acidic phosphates include the phosphates listed hereinabove.

Basic recognition elements include amines. Suitable basic amines include alkyl amines, aryl amines, aryl alkyl amines, pyridines, heterocyclic amines (saturated or unsaturated, the nitrogen in the ring or not), amidines, and any additional amines listed hereinabove. Suitable alkyl amines include that of formula B9. Suitable heterocyclic or alkyl heterocyclic amines include that of formula A9. Suitable pyridines include those of formulas A5 and B5.

Recognition elements including a hydrogen bond donor include amines, amides, carboxyls, protonated phosphates, protonated phosphonates, protonated phosphinates, protonated sulphates, protonated sulphinates, alcohols, and thiols. Suitable amines include alkyl amines, aryl amines, aryl alkyl amines, pyridines, heterocyclic amines (saturated or unsaturated, the nitrogen in the ring or not), amidines, ureas, and any other amines listed hereinabove. Suitable alkyl amines include that of formula B9. Suitable heterocyclic or alkyl heterocyclic amines include that of formula A9. Suitable pyridines include those of formulas A5 and B5. Suitable protonated carboxylates, protonated phosphates include those listed hereinabove. Suitable amides include those of formulas A8 and B8. Suitable alcohols include primary alcohols, secondary alcohols, tertiary alcohols, and aromatic alcohols (e.g., phenols). Suitable alcohols include those of formulas A7 (a primary alcohol) and B7 (a secondary alcohol).

Recognition elements including a hydrogen bond acceptor or one or more free electron pairs include amines, amides, carboxylates, carboxyl groups, phosphates, phosphonates, phosphinates, sulphates, sulphonates, alcohols, ethers, thiols, and thioethers. Suitable amines include alkyl amines, aryl amines, aryl alkyl amines, pyridines, heterocyclic amines (saturated or unsaturated, the nitrogen in the ring or not), amidines, ureas, and amines as listed hereinabove. Suitable alkyl amines include that of formula B9. Suitable heterocyclic or alkyl heterocyclic amines include that of formula A9. Suitable pyridines include those of formulas A5 and B5. Suitable carboxylates include those listed hereinabove. Suitable amides include those of formulas A8 and B8. Suitable phosphates, phosphonates and phosphinates include those listed hereinabove. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include alkyl ethers, aryl alkyl ethers. Suitable alkyl ethers include that of formula A6. Suitable aryl alkyl ethers include that of formula A4. Suitable thioethers include that of formula B6.

Recognition elements including uncharged polar or hydrophilic groups include amides, alcohols, ethers, thiols, thioethers, esters, thio esters, boranes, borates, and metal complexes. Suitable amides include those of formulas A8 and B8. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include those listed hereinabove. Suitable ethers include that of formula A6. Suitable aryl alkyl ethers include that of formula A4.

Recognition elements including uncharged hydrophobic groups include alkyl (substituted and unsubstituted), alkene (conjugated and unconjugated), alkyne (conjugated and unconjugated), aromatic. Suitable alkyl groups include lower alkyl, substituted alkyl, cycloalkyl, aryl alkyl, and heteroaryl alkyl. Suitable lower alkyl groups include those of formulas A1, A3, A3a, and B1. Suitable aryl alkyl groups include those of formulas A3, A3a, A4, B3, B3a, and B4. Suitable alkyl cycloalkyl groups include that of formula B2. Suitable alkene groups include lower alkene and aryl alkene. Suitable aryl alkene groups include that of formula B4. Suitable aromatic groups include unsubstituted aryl, heteroaryl, substituted aryl, aryl alkyl, heteroaryl alkyl, alkyl substituted aryl, and polyaromatic hydrocarbons. Suitable aryl alkyl groups include those of formulas A3, A3a and B4. Suitable alkyl heteroaryl groups include those of formulas A5 and B5.

Spacer (e.g., small) recognition elements include hydrogen, methyl, ethyl, and the like. Bulky recognition elements include 7 or more carbon or hetero atoms.

Formulas A1-A9 and B1-B9 are:

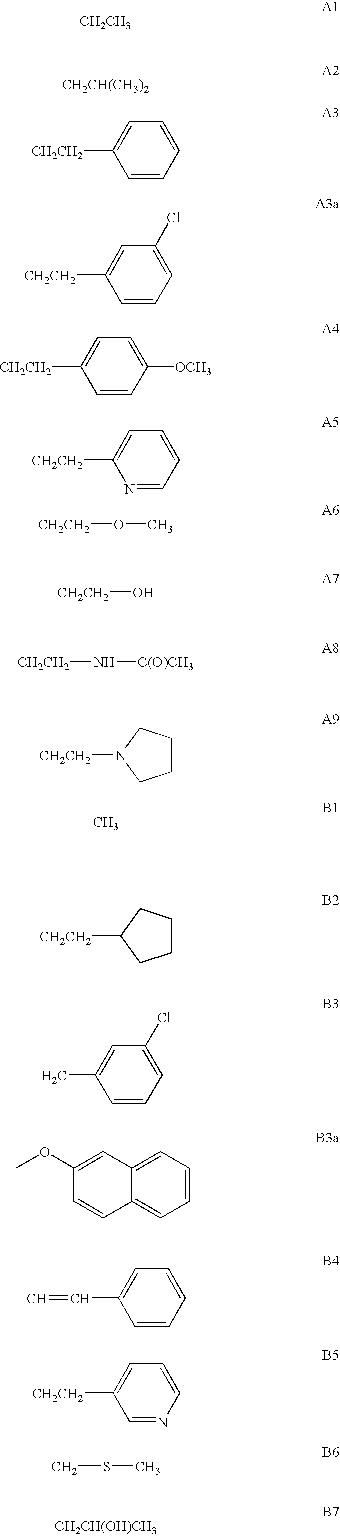

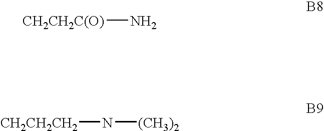

These A and B recognition elements can be called derivatives of, according to a standard reference: A1, ethylamine; A2, isobutylamine; A3, phenethylamine; A4, 4-methoxyphenethylamine; A5,2-(2-aminoethyl)pyridine; A6,2-methoxyethylamine; A7, ethanolamine; A8, N-acetylethylenediamine; A9, 1-(2-aminoethyl)pyrrolidine; B1, acetic acid, B2, cyclopentylpropionic acid; B3,3-chlorophenylacetic acid; B4, cinnamic acid; B5,3-pyridinepropionic acid; B6, (methylthio)acetic acid; B7,3-hydroxybutyric acid; B8, succinamic acid; and B9,4-(dimethylamino)butyric acid.

In an embodiment, the A recognition elements are linked to a framework at a pendant position. In an embodiment, the B recognition elements are linked to a framework at an equatorial position. In an embodiment, the A recognition elements are linked to a framework at a pendant position and the B recognition elements are linked to the framework at an equatorial position.

In an embodiment, the building blocks including the A and B recognition elements can be visualized as occupying a binding space defined by lipophilicity/hydrophilicity and volume. A volume can be calculated (using known methods) for each building block including the various A and B recognition elements. A measure of lipophilicity/hydrophilicity (log P) can be calculated (using known methods) for each building block including the various A and B recognition elements. Negative values of log P show affinity for water over nonpolar organic solvent and indicate a hydrophilic nature. A plot of volume versus log P can then show the distribution of the building blocks through a binding space defined by size and lipophilicity/hydrophilicity.

Reagents that form many of the recognition elements are commercially available. For example, reagents for forming recognition elements A1, A2, A3, A3a, A4, A5, A6, A7, A8, A9 B1, B2, B3, B3a, B4, B5, B6, B7, B8, and B9 are commercially available.

### Linkers

The linker is selected to provide a suitable coupling of the building block to a support. The framework can interact with the ligand as part of the artificial receptor. The linker can also provide bulk, distance from the support, hydrophobicity, hydrophilicity, and like structural characteristics to the building block. Coupling building blocks to the support can employ covalent bonding or noncovalent interactions. Suitable noncovalent interactions include interactions between ions, hydrogen bonding, van der Waals interactions, and the like. In an embodiment, the linker includes moieties that can engage in covalent bonding or noncovalent interactions. In an embodiment, the linker includes moieties that can engage in covalent bonding. Suitable groups for forming covalent and reversible covalent bonds are described hereinabove.

The linker can be selected to provide a suitable covalent coupling of the building block to a support. The framework can interact with the ligand as part of the artificial receptor. The linker can also provide bulk, distance from the support, hydrophobicity, hydrophilicity, and like structural characteristics to the building block. In an embodiment, the linker forms a covalent bond with a functional group on the framework. In an embodiment, before attachment to the support the linker also includes a functional group that can be activated to react with or that will react with a functional group on the support. In an embodiment, once attached to the support, the linker forms a covalent bond with the support and with the framework.

In an embodiment, the linker forms or can be visualized as forming a covalent bond with an alcohol, phenol, thiol, amine, carbonyl, or like group on the framework. The linker can include a carboxyl, alcohol, phenol, thiol, amine, carbonyl, maleimide, or like group that can react with or be activated to react with the support. Between the bond to the framework and the group formed by the attachment to the support, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

The linker can include a good leaving group bonded to, for example, an alkyl or aryl group. The leaving group being “good” enough to be displaced by the alcohol, phenol, thiol, amine, carbonyl, or like group on the framework. Such a linker can include a moiety represented by the formula: R-X, in which X is a leaving group such as halogen (e.g., —Cl, —Br or —I), tosylate, mesylate, triflate, and R is alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

Suitable linker groups include those of formula: (CH₂)ₙCOOH, with n=1-16, n=2-8, n=2-6, or n=3. Reagents that form suitable linkers are commercially available and include any of a variety of reagents with orthogonal functionality.

### Embodiments of Building Blocks

In an embodiment, building blocks can be represented by Formula 2:

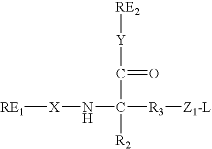

in which: RE₁ is recognition element 1, RE₂ is recognition element 2, and L is a linker. X is absent, C═O, CH₂, NR, NR₂, NH, NHCONH, SCONH, CH═N, or OCH₂NH. In certain embodiments, X is absent or C═O. Y is absent, NH, O, CH₂, or NRCO. In certain embodiments, Y is NH or O. In an embodiment, Y is NH. Z₁ and Z₂ can independently be CH2, O, NH, S, CO, NR, NR₂, NHCONH, SCONH, CH═N, or OCH₂NH. In an embodiment, Z₁ and/or Z₂ can independently be O. Z₂ is optional. R₂ is H, CH₃, or another group that confers chirality on the building block and has size similar to or smaller than a methyl group. R₃ is CH₂; CH₂-phenyl; CHCH₃; (CH₂)ₙ with n=2-3; or cyclic alkyl with 3-8 carbons, e.g., 5-6 carbons, phenyl, naphthyl. In certain embodiments, R₃ is CH₂ or CH₂-phenyl.

RE₁ is B1, B2, B3, B3a, B4, B5, B6, B7, B8, B9, A1, A2, A3, A3a, A4, A5, A6, A7, A8, or A9. In certain embodiments, RE₁ is B1, B2, B3, B3a, B4, B5, B6, B7, B8, or B9. RE₂ is A1, A2, A3, A3a, A4, A5, A6, A7, A8, A9, B1, B2, B3, B3a, B4, B5, B6, B7, B8, or B9. In certain embodiments, RE₂ is A1, A2, A3, A3a, A4, A5, A6, A7, A8, or A9. In an embodiment, RE₁ can be B2, B3a, B4, B5, B6, B7, or B8. In an embodiment, RE₂ can be A2, A3a, A4, A5, A6, A7, or A8.

In an embodiment, L is the functional group participating in or formed by the bond to the framework (such groups are described herein), the functional group or groups participating in or formed by the reversible interaction with the support or lawn (such groups are described herein), and a linker backbone moiety. In an embodiment, the linker backbone moiety is about 4 to about 48 carbon or heteroatom alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or mixtures thereof; or about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms.

In an embodiment, the L is the functional group participating in or formed by the bond to the framework (such groups are described herein) and a lipophilic moiety (such groups are described herein) of about 4 to about 48 carbons, about 8 to about 14 carbons, about 12 to about 24 carbons, about 16 to about 18 carbons. In an embodiment, this L also includes about 1 to about 8 reversible bond/interaction moieties (such groups are described herein) or about 2 to about 4 reversible bond/interaction moieties. In an embodiment, L is (CH₂)ₙCOOH, with n=12-24, n=17-24, or n=16-18.

In an embodiment, L is (CH₂)ₙCOOH, with n=1-16, n=2-8, n=4-6, or n=3.

Building blocks including an A and/or a B recognition element, a linker, and an amino acid framework can be made by methods illustrated in general Scheme 1.

### Methods of Making an Artificial Receptor

The present invention relates to a method of making an artificial receptor or a candidate artificial receptor. In an embodiment, this method includes preparing a spot or region on a support, the spot or region including a plurality of building blocks immobilized on the support. The method can include mixing a plurality of building blocks and employing the mixture in forming the spot(s). Alternatively, the method can include spotting individual building blocks on the support. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in covalent bonding or noncovalent interactions. The method can apply or spot building blocks onto a support in combinations of 2, 3, 4, or more building blocks.

In an embodiment, the present method includes making a receptor surface. Making a receptor surface can include forming a region on a solid support, the region including a plurality of building blocks, and immobilizing the plurality of building blocks to the solid support in the region. The method can include mixing a plurality of building blocks and employing the mixture in forming the region or regions. Alternatively, the method can include applying individual building blocks in a region on the support. Forming a region on a support can be accomplished, for example, by soaking a portion of the support with the building block solution. The resulting coating including building blocks can be referred to as including heterogeneous building blocks.

In an embodiment, the method produces a spot or surface with a density of building blocks sufficient to provide interactions of more than one building block with a ligand. That is, the building blocks can be in proximity to one another. Proximity of different building blocks can be detected by determining different (e.g., greater) binding of a ligand of interest to a spot or surface including a plurality of building blocks compared to a spot or surface including only one of the building blocks.

The method can immobilize building blocks on supports using known methods for immobilizing compounds of the types employed as building blocks. Coupling building blocks to the support can employ covalent bonding or noncovalent interactions. In an embodiment, the support can be functionalized with moieties that can engage in covalent bonding, e.g., reversible covalent bonding. The present invention can employ any of a variety of the numerous known functional groups, reagents, and reactions for forming reversible covalent bonds. Suitable reagents for forming reversible covalent bonds include those described in Green, T W; Wuts, P G M (1999), Protective Groups in Organic Synthesis Third Edition, Wiley-Interscience, New York, 779 pp. For example, the support can include functional groups such as a carbonyl group, a carboxyl group, a silane group, boric acid or ester, an amine group (e.g., a primary, secondary, or tertiary amine, a hydroxylamine, a hydrazine, or the like), a thiol group, an alcohol group (e.g., primary, secondary, or tertiary alcohol), a diol group (e.g., a 1,2 diol or a 1,3 diol), a phenol group, a catechol group, or the like. These functional groups can form groups with reversible covalent bonds, such as ether (e.g., alkyl ether, silyl ether, thioether, or the like), ester (e.g., alkyl ester, phenol ester, cyclic ester, thioester, or the like), acetal (e.g., cyclic acetal), ketal (e.g., cyclic ketal), silyl derivative (e.g., silyl ether), boronate (e.g., cyclic boronate), amide, hydrazide, imine, carbamate, or the like. Such a functional group can be referred to as a covalent bonding moiety, e.g., a first covalent bonding moiety.

A carbonyl group on the support and an amine group on a building block can form an imine or Schiff's base. The same is true of an amine group on the support and a carbonyl group on a building block. A carbonyl group on the support and an alcohol group on a building block can form an acetal or ketal. The same is true of an alcohol group on the support and a carbonyl group on a building block. A thiol (e.g., a first thiol) on the support and a thiol (e.g., a second thiol) on the building block can form a disulfide.

A carboxyl group on the support and an alcohol group on a building block can form an ester. The same is true of an alcohol group on the support and a carboxyl group on a building block. Any of a variety of alcohols and carboxylic acids can form esters that provide covalent bonding that can be reversed in the context of the present invention.

For example, reversible ester linkages can be formed from alcohols such as phenols with electron withdrawing groups on the aryl ring, other alcohols with electron withdrawing groups acting on the hydroxyl-bearing carbon, other alcohols, or the like; and/or carboxyl groups such as those with electron withdrawing groups acting on the acyl carbon (e.g., nitrobenzylic acid, R—CF₂—COOH, R—CCl₂—COOH, and the like), other carboxylic acids, or the like.

### Test Ligands

The test ligand can be any ligand for which binding to an array or surface can be detected. The test ligand can be a pure compound, a mixture, or a “dirty” mixture containing a natural product or pollutant. Such dirty mixtures can be tissue homogenate, biological fluid, soil sample, water sample, or the like.

Test ligands include prostate specific antigen, other cancer markers, insulin, warfarin, other anti-coagulants, cocaine, other drugs-of-abuse, markers for E. coli, markers for Salmonella sp., markers for other food-borne toxins, food-borne toxins, markers for Smallpox virus, markers for anthrax, markers for other possible toxic biological agents, pharmaceuticals and medicines, pollutants and chemicals in hazardous waste, toxic chemical agents, markers of disease, pharmaceuticals, pollutants, biologically important cations (e.g., potassium or calcium ion), peptides, carbohydrates, enzymes, bacteria, viruses, mixtures thereof, and the like. In certain embodiments, the test ligand can be at least one of small organic molecules, inorganic/organic complexes, metal ion, mixture of proteins, protein, nucleic acid, mixture of nucleic acids, mixtures thereof, and the like.

Suitable test ligands include any compound or category of compounds described elsewhere in this document as being a test ligand, including, for example, the microbes, proteins, cancer cells, drugs of abuse, and the like described above.

### EXAMPLES

### Example 1

### Competition Between A Conjugate and Glucose

Competition between a sugar-dendrimer conjugate and free glucose for a combinatorial artificial receptor yielded a significant change in an optical signal.

### Materials and Methods

Dendrimers of poly(amidoamine) on an ethylenediamine core, PAMAM dendrimers, were obtained from DENDRITECH®, Inc. One such dendrimer is illustrated in Scheme I, below.

Conjugates including a sugar and a dendrimer like the one illustrated in Scheme I were prepared as described in Scheme II, below.

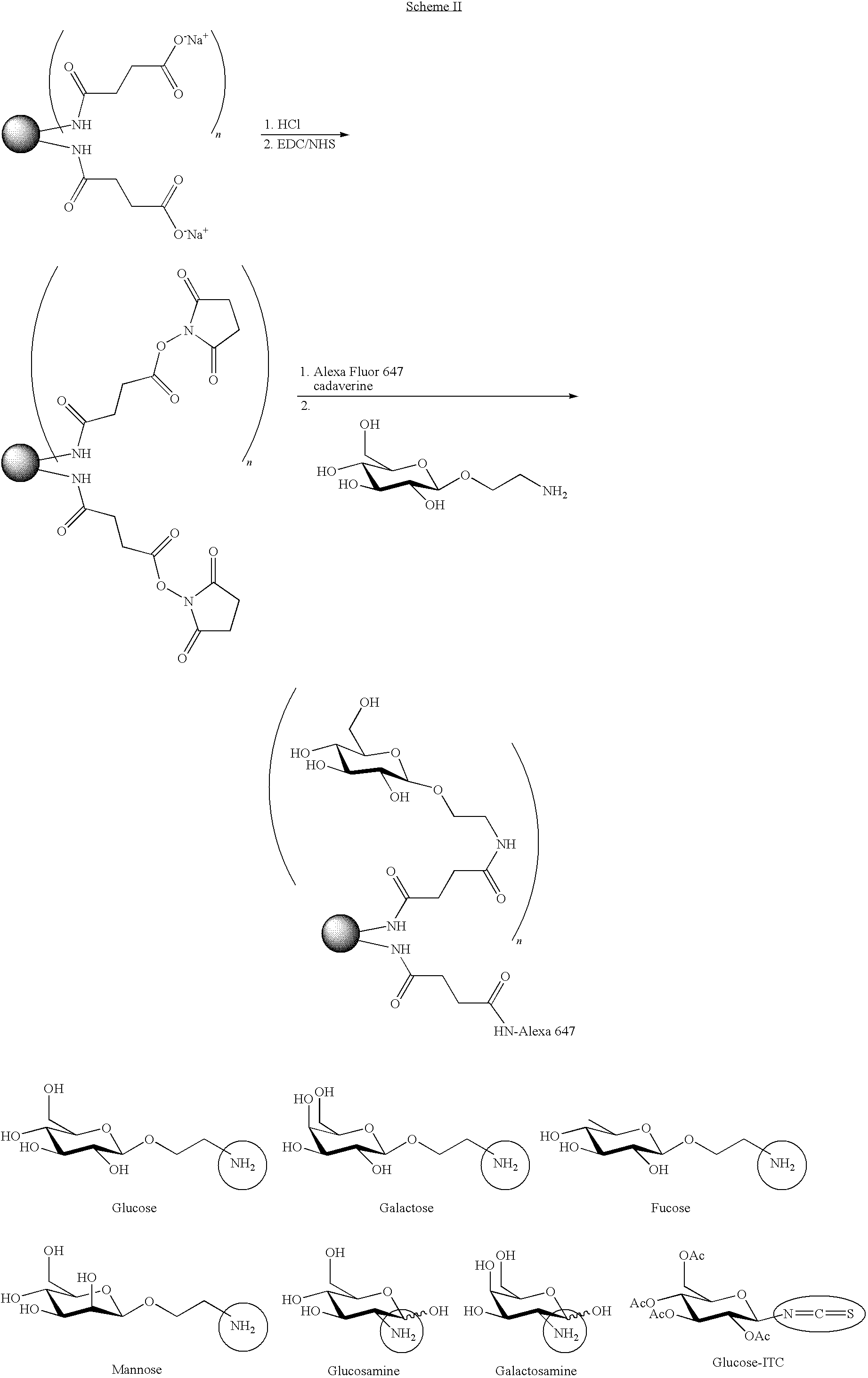

The a conjugate of the amino-glucose derivative shown in Scheme II and a 4.5 generation PAMAM dendrimer was prepared and evaluated against an array containing candidate artificial receptors.

### Results and Discussion

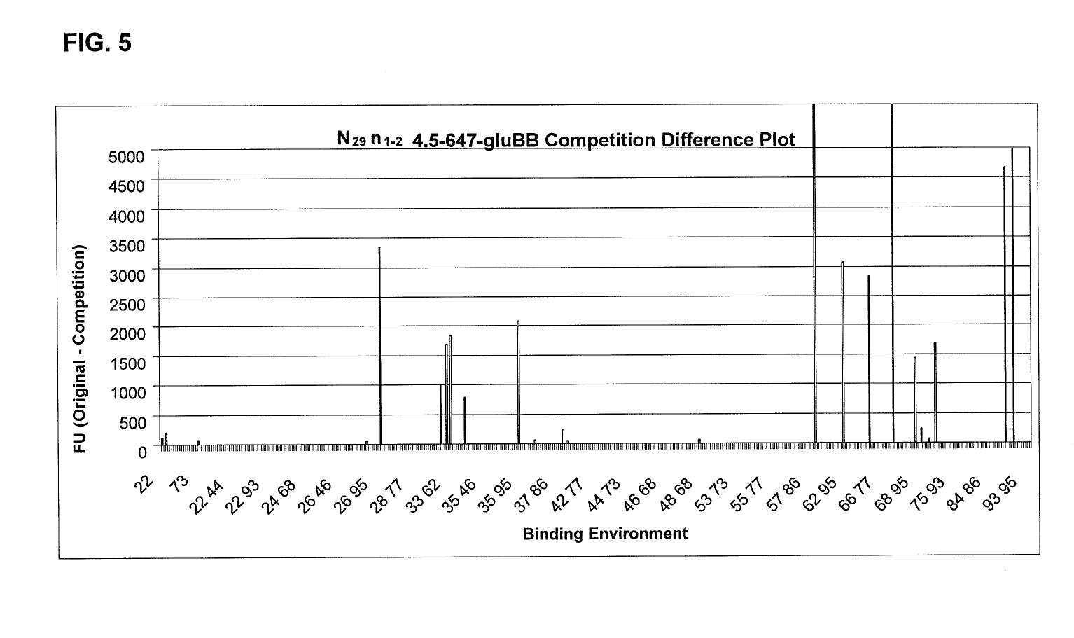

The array was constructed from 29 building blocks in homogeneous spots and in combinations of two building blocks. Those building blocks that gave competitive binding alone or in combinations of two building blocks were selected as likely to give rise to robust, tunable competitive binding in combinations including a greater number of building blocks. FIG. 5 illustrates the results of this study as fluorescence change upon addition of competitor against identity of building blocks in the candidate artificial receptor. Those candidate receptors that provided a detectable difference in binding of the conjugate in the presence and absence of glucose were selected for further study.

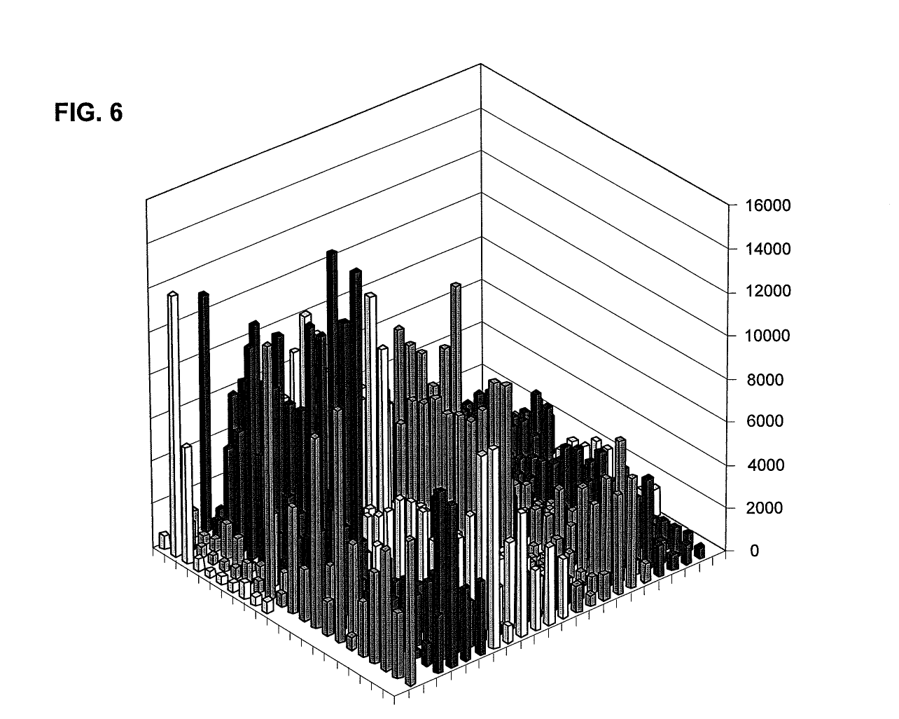

This further study was conducted using nine building blocks in homogeneous receptors and in candidate receptors including combinations of two to nine building blocks. FIG. 6 illustrates the results obtained for binding of the glucose-dendrimer conjugate to these candidate artificial receptors. As illustrated, receptors including three to six building blocks, for example, 3, 4, or 5 building blocks, provided the most diverse binding—highs and lows in FIG. 6—which is useful for obtaining the desired receptor development.

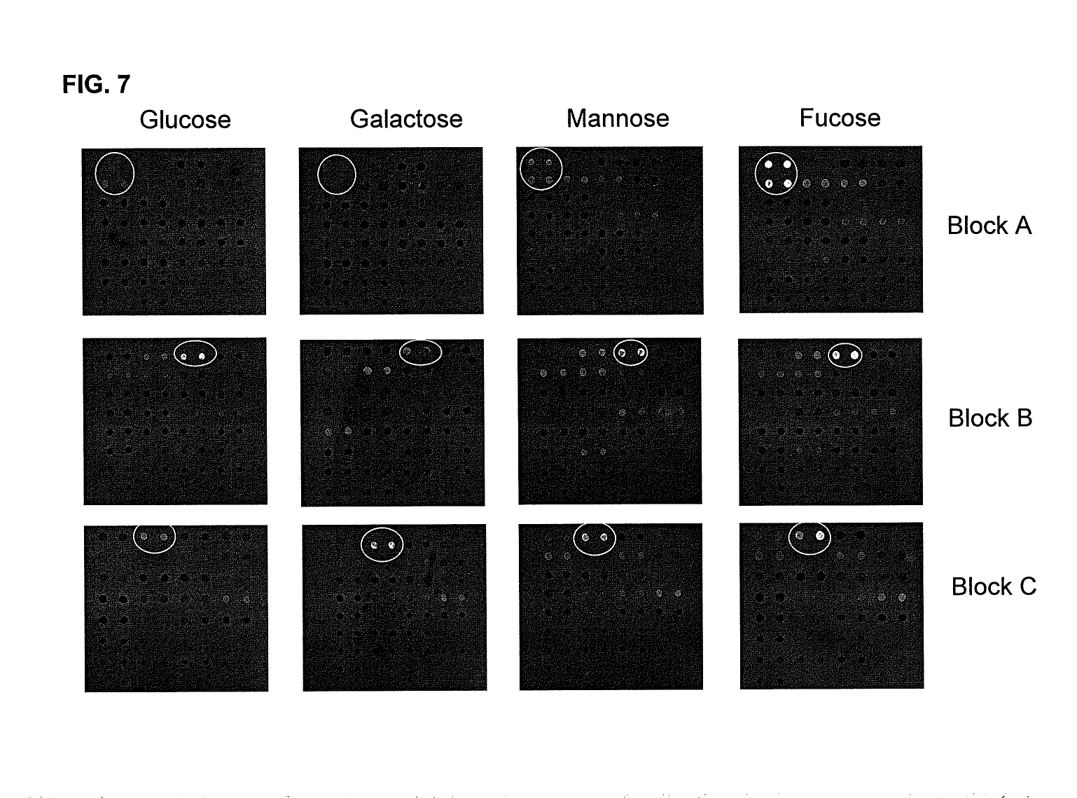

FIG. 7 illustrates the binding to candidate artificial receptors obtained using conjugates of the dendrimer with each of three additional sugars, galactose, mannose, and fucose. In FIG. 7, each block is a group of candidate receptors from an array. The circled receptors represent those that respond differently to the different conjugates.

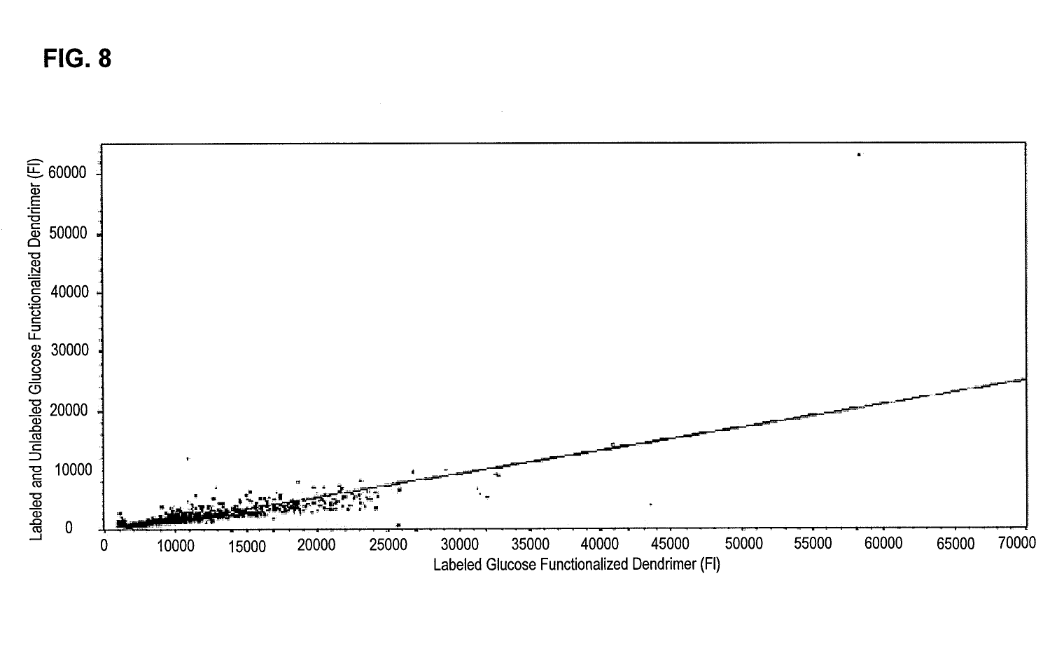

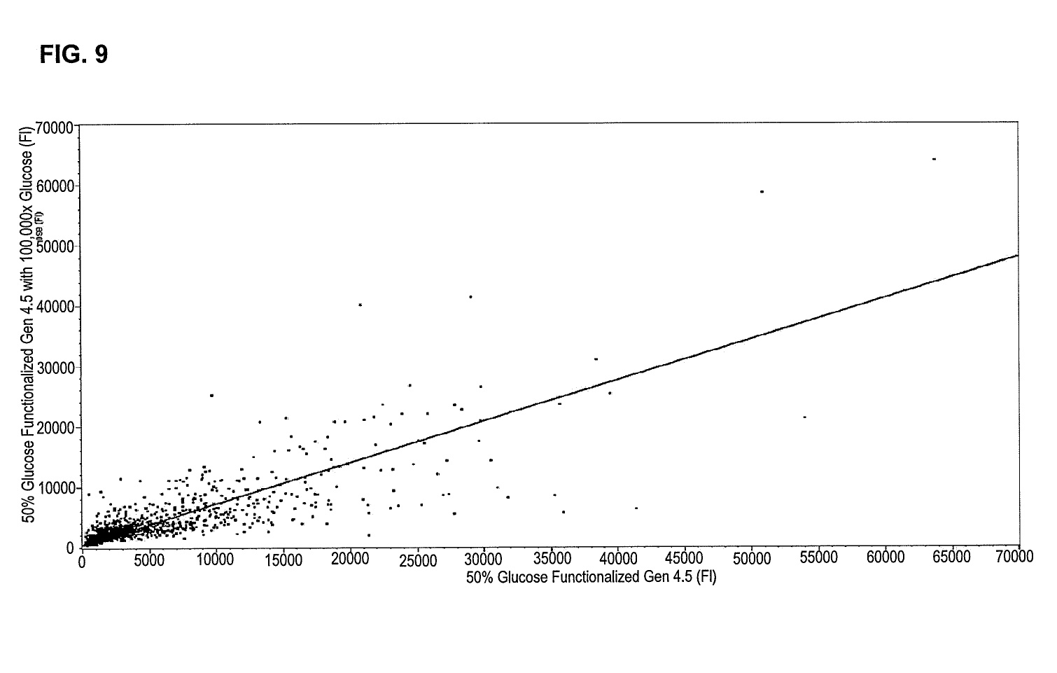

FIGS. 8 and 9 illustrate the results of competition for these candidate receptors. In the study reported in FIG. 8, the labeled conjugate of glucose and dendrimer competed against the unlabeled conjugate for each of the candidate artificial receptors. The unlabeled conjugate was at a concentration 100-times the concentration of the labeled conjugate. In the absence of competition, the points in a graph like FIG. 8 would have clustered around a line with a slope of one (which would extend from the origin of the graph to its upper right corner). As shown in FIG. 8, the unlabeled conjugate competed with the labeled conjugate and the points define a line with a slope significantly less than one.

FIG. 9 illustrates the results of an experiment in which the labeled conjugate of glucose and dendrimer competed with glucose. In the absence of competition, the points in a graph like FIG. 9 would have clustered around a line with a slope of one (which would extend from the origin of the graph to its upper right corner). In fact, the labeled conjugate of glucose and dendrimer competed with glucose at a range of levels, as depicted by the span of points shown in FIG. 9. A best fit line of the points has a slope of less than one, indicating competition, but the range of competition levels across the different binding environments demonstrates the adaptability of the system. Different binding environments gave rise to different levels of competition between glucose and the labeled conjugate of glucose and dendrimer. Those candidate receptors that provided the highest level of competition include those represented by data points that are significantly beneath the line in FIG. 9 and that correspond to a value along the x-axis of, for example, 25,000 to 45,000 fluorescence units. However all receptors that showed competitive binding are candidates and the range of binding responses generated in this experiment can be used to tune the system's response to glucose.

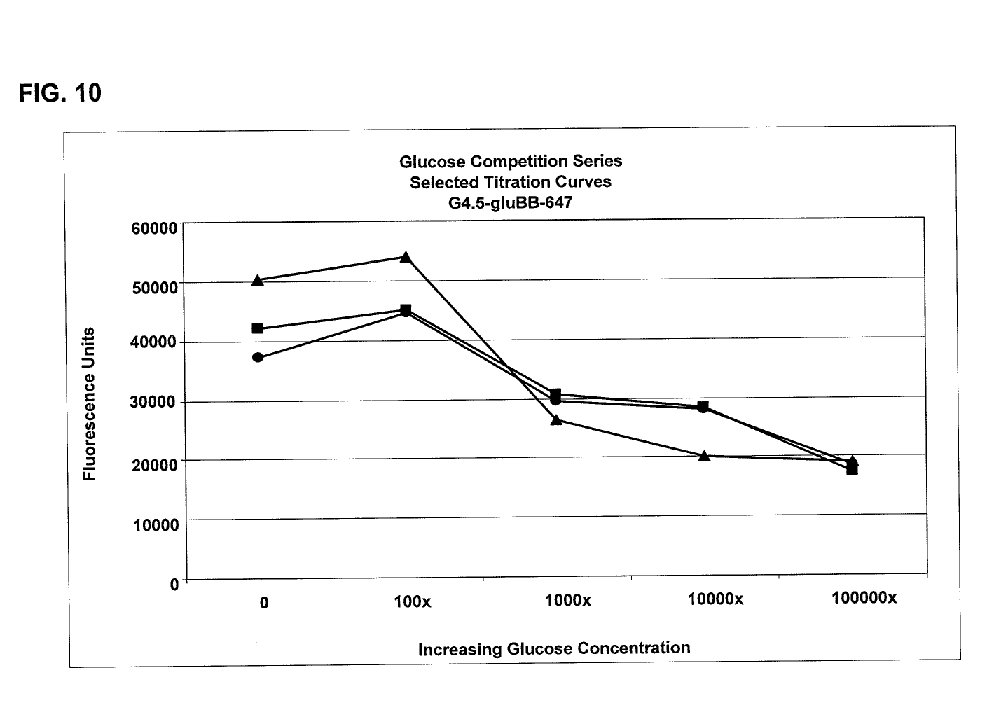

FIG. 10 illustrates the decrease in fluorescence from bound labeled conjugate that was obtained upon competition with glucose at three of the candidate artificial receptors. Receptor A (triangles on graph) included building block TyrA₉B₃. Receptor B (squares on graph) included building blocks TyrA₂B₂, TyrA₃B₇, TyrA₄B₂, TyrA₉B₁, and TyrA₉B₃. Receptor C (circles on graph) included building blocks TyrA₄B₂, TyrA₅B₃, TyrA₇B₃, TyrA₉B₁, and TyrA₉B₃.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A detector system comprising:
a competitor comprising an analog of a ligand of interest covalently coupled to a macromolecule;
an artificial receptor configured to bind a ligand of interest and the competitor, binding of the ligand of interest competing with binding of the competitor;
the artificial receptor comprising a plurality of different building block molecules independently covalently coupled to a solid support in a region; the region being a contiguous portion of the surface of the solid support with the different building block molecules distributed randomly throughout the contiguous region;
a semipermeable membrane that is permeable to the ligand of interest but that retains an effective amount of the competitor;
the semipermeable membrane and the artificial receptor at least partially defining a chamber, the competitor and the artificial receptor being in the chamber; and
a detector operatively coupled to the artificial receptor and configured to, in response to binding of the competitor or the ligand of interest to the artificial receptor, produce indicia of presence or concentration of the ligand of interest.

2. The system of claim 1, wherein the ligand of interest is glucose, the macromolecule comprises a dendrimer, and the artificial receptor is an artificial glucose receptor.

3. The system of claim 1, wherein:
the artificial glucose receptor and the competitor are configured to provide competition of glucose and competitor for the artificial glucose receptor at concentrations of glucose that are achieved in the detector system when the detector system is exposed to a physiological concentration of glucose; and
the detector is configured provide indicia of a low concentration of glucose, an acceptable concentration of glucose, and a high concentration of glucose.

4. The system of claim 1, wherein the competitor further comprises a detectable label covalently bonded to the macromolecule.

5. The system of claim 1, wherein the macromolecule is a dendrimer.

6. The system of claim 1, further comprising isotonic liquid filling the chamber.

7. The system of claim 1, wherein the detector comprises an optical detector.

8. The system of claim 1, wherein the detector comprises a quartz crystal microbalance.

9. The system of claim 1, wherein the detector comprises an antenna and an integrated circuit, the integrated circuit being configured to store the indicia, receive a first radio signal, and transmit a second radio signal; the first radio signal providing power to activate the integrated circuit to transmit the second radio signal; the second radio signal comprising the indicia.

10. A method of determining a level of a ligand of interest, comprising:
implanting in a subject a detector system;
retrieving from the detector system indicia of presence or concentration of the ligand of interest;
the detector system comprising:
a competitor comprising an analog of a ligand of interest covalently coupled to a macromolecule;
an artificial receptor configured to bind a ligand of interest and the competitor, binding of the ligand of interest competing with binding of the competitor;
the artificial receptor comprising a plurality of different building block molecules independently covalently coupled to a solid support in a region; the region being a contiguous portion of the surface of the solid support with the different building block molecules distributed randomly throughout the contiguous region;
a semipermeable membrane that is permeable to the ligand of interest but that retains an effective amount of the competitor;
the semipermeable membrane and the artificial receptor at least partially defining a chamber, the competitor and the artificial receptor being in the chamber; and
a detector operatively coupled to the artificial receptor and configured to, in response to binding of the competitor or the ligand of interest to the artificial receptor, produce indicia of presence or concentration of the ligand of interest.

11. The method of claim 10, wherein the ligand of interest is glucose; and
implanting comprises implanting the detector system in a subject so that it is in fluid communication with a biological fluid that contains a level of glucose indicative of blood glucose levels.

12. The method of claim 11, comprising implanting the detector system in a blood vessel, in muscle, or in skin.

13. The method of claim 11, wherein:
the artificial glucose receptor and the competitor are configured to provide competition of glucose and competitor for the artificial glucose receptor at concentrations of glucose that are achieved in the detector system when the detector system is exposed to a physiological concentration of glucose; and
the detector is configured provide indicia of a low concentration of glucose, an acceptable concentration of glucose, and a high concentration of glucose.

14. The method of claim 13, wherein the detector system is effective to indicate glucose level in the presence of about 80 to about 120 mg/dL glucose, 2 to 12 mg/dL fructose, and 1.5 to 90 mg/dL galactose.

15. The method of claim 10, wherein the detector comprises an antenna and an integrated circuit, the integrated circuit being configured to store the indicia, receive a first radio signal, and transmit a second radio signal; the first radio signal providing power to activate the integrated circuit to transmit the second radio signal; the second radio signal comprising the indicia.

16. The method of claim 15, wherein retrieving the indicia comprises disposing a reader proximal the implanted detector system;
the reader being configured to transmit the first radio signal; to receive the second radio signal; and to display information regarding the presence or concentration of the ligand of interest.

17. An artificial receptor system comprising:
a molecular competitor that competes against binding of a ligand of interest;
an artificial receptor configured to bind a ligand of interest and the competitor, binding of the ligand of interest competing with binding of the competitor; the artificial receptor comprising a plurality of different building block molecules independently covalently coupled to a solid support;
a ligand permeable interface that is permeable to the ligand of interest but that retains an effective amount of the competitor; the ligand permeable interface and the artificial receptor at least partially defining a chamber, the competitor and the artificial receptor being in the chamber; and
a detector operatively coupled to the artificial receptor and configured to produce indicia of presence or concentration of the ligand of interest.

18. The system of claim 17, wherein the ligand of interest is glucose, the competitor comprises a dendrimer, and the artificial receptor is an artificial glucose receptor.

19. The system of claim 18, wherein:
the artificial glucose receptor and the competitor are configured to provide competition of glucose and competitor for the artificial glucose receptor at concentrations of glucose that are achieved in the detector system when the detector system is exposed to a physiological concentration of glucose; and
the detector is configured provide indicia of a low concentration of glucose, an acceptable concentration of glucose, and a high concentration of glucose.

20. The system of claim 17, further comprising isotonic liquid filling the chamber.

21. The system of claim 17, wherein the detector comprises an antenna and an integrated circuit, the integrated circuit being configured to store the indicia, receive a first radio signal, and transmit a second radio signal; the first radio signal providing power to activate the integrated circuit to transmit the second radio signal; the second radio signal comprising the indicia.

22. A method of determining a level of a ligand of interest, comprising:
implanting in a subject a detector system;
retrieving from the detector system indicia of presence or concentration of the ligand of interest;
the detector system comprising:
a molecular competitor that competes against binding of a ligand of interest;
an artificial receptor configured to bind a ligand of interest and the competitor, binding of the ligand of interest competing with binding of the competitor; the artificial receptor comprising a plurality of different building block molecules independently covalently coupled to a solid support;
a ligand permeable interface that is permeable to the ligand of interest but that retains an effective amount of the competitor; the ligand permeable interface and the artificial receptor at least partially defining a chamber, the competitor and the artificial receptor being in the chamber; and
a detector operatively coupled to the artificial receptor and configured to produce indicia of presence or concentration of the ligand of interest.

23. The method of claim 22, wherein the ligand of interest is glucose; and
implanting comprises implanting the detector system in a subject so that it is in fluid communication with a biological fluid that contains a level of glucose indicative of blood glucose levels.

24. The method of claim 23, comprising implanting the detector system in a blood vessel, in muscle, or in skin.

25. The method of claim 23, wherein:
the artificial glucose receptor and the competitor are configured to provide competition of glucose and competitor for the artificial glucose receptor at concentrations of glucose that are achieved in the detector system when the detector system is exposed to a physiological concentration of glucose; and
the detector is configured provide indicia of a low concentration of glucose, an acceptable concentration of glucose, and a high concentration of glucose.

26. The method of claim 25, wherein the detector system is effective to indicate glucose level in the presence of about 80 to about 120 mg/dL glucose, 2 to 12 mg/dL fructose, and 1.5 to 90 mg/dL galactose.

27. The method of claim 22, wherein the detector comprises an antenna and an integrated circuit, the integrated circuit being configured to store the indicia, receive a first radio signal, and transmit a second radio signal; the first radio signal providing power to activate the integrated circuit to transmit the second radio signal; the second radio signal comprising the indicia.

28. The method of claim 27, wherein retrieving the indicia comprises disposing a reader proximal the implanted detector system;
the reader being configured to transmit the first radio signal; to receive the second radio signal; and to display information regarding the presence or concentration of the ligand of interest.

29. An artificial receptor system comprising:
a competitor comprising an analog of a ligand of interest covalently coupled to a macromolecule;
an artificial receptor configured to bind a ligand of interest and the competitor, binding of the ligand of interest competing with binding of the competitor;
the artificial receptor comprising a plurality of different building block molecules independently covalently coupled to a solid support in a region; the region being a contiguous portion of the surface of the solid support with the different building block molecules distributed randomly throughout the contiguous region.

30. An artificial receptor system comprising:
a molecular competitor that competes against binding of a ligand of interest;
an artificial receptor configured to bind a ligand of interest and the competitor, binding of the ligand of interest competing with binding of the competitor; the artificial receptor comprising a plurality of different building block molecules independently covalently coupled to a solid support.
